(19) United States
(12) Patent Application Publication
(10) Pub. No.: US 2026/0146260 A1
(43) Pub. Date: May 28, 2026

(54) Transduction of gammadelta T cells with pseudotyped retroviral vectors

(71) Applicant: Miltenyi Biotec B.V. & Co. KG, Bergisch Gladbach (DE)

(72) Inventors: Jose Alberto Villacorta Hidalgo, Bergisch Gladbach (DE); Lorraine Pinot, Bergisch Gladbach (DE); Nina Moker, Bergisch Gladbach (DE)

(73) Assignee: Miltenyi Biotec B.V. & Co. KG, Bergisch Gladbach (DE)

(21) Appl. No.: 19/121,097

(22) PCT Filed: Oct. 6, 2023
(86) PCT No.: PCT/EP2023/077737

§ 371 Date: Apr. 14, 2025

(30) Foreign Application Priority Data
Oct. 15, 2022 (EP) 22201758.4

(51) Int. Cl. C12N 15/867; C07K 14/15; C07K 14/725; C12N 5/0783; C12N 5/10

(52) U.S. Cl. CPC C12N 15/867; C07K 14/15; C07K 14/7051; C12N 5/0636; C12N 5/10; C07K 2319/03; C07K 2319/33; C12N 2500/46; C12N 2500/99; C12N 2501/2302; C12N 2501/2315; C12N 2510/00; C12N 2740/10023; C12N 2740/10043

(57) ABSTRACT

The present invention provides an in-vitro method for transferring one or more nucleic acids sequences comprising one or more transgenes into γδ T cells with a pseudotyped retroviral vector particle or a virus-like particle thereof, wherein said pseudotyped retroviral vector particle or virus-like particle thereof comprises a modified baboon endogenous retrovirus (BaEV) envelope glycoprotein, the method comprising the steps a) activation of γδ T cells, b) contacting said pseudotyped retroviral vector particle or virus-like particle thereof with said activated γδ T cells using a low concentration of said pseudotyped retroviral vector particle, c) expanding said genetically modified γδ T cells in the absence of an aminobisphosphonate and in the presence of IL-2 and IL-15, wherein said expansion is in the absence of feeder cells and in the absence of human serum, and wherein at least 75% of the transduced and expanded γδ T cells are CD45RA-γδ T cells.

### FIELD OF THE INVENTION

The present invention relates to the field of transduction of T cells, in particular to the transduction of γδ T cells with a retroviral vector pseudotyped with a modified baboon endogenous retrovirus (BaEV) envelope glycoprotein.

### BACKGROUND OF THE INVENTION

γδ T-cells have been identified infiltrating distinct cancers as an interesting part of the tumor microenvironment with the demonstration of cytotoxicity in-vitro against both solid and hematological malignancies (1,2,3). γδ T cells represent a small population of T lymphocytes in human adults, present in peripheral blood and tissues. Vγ9Vδ2 T lymphocytes are the main subset in the human adult peripheral blood, where γδ T-cells typically constitute about 5% of CD3+ lymphocytes. In addition to Vγ9Vδ2 T-cells, lymphocytes expressing Vδ1 are typically enriched in human tissues such as the intestine, mucosae, and skin (4, 5). Even though they constitute only a small population of lymphocytes, γδ T-cells may play a non-overlapping role in some human infections, autoimmunity (6), and tumor microenvironment (7). The Vγ9Vδ2 T-cell subset recognizes phosphoantigens such as isopentenyl pyrophosphate (IPP). IPP is produced in all higher eukaryotic cells including human cancer cells by the mevalonate pathway. In contrast, many bacteria such as Mycobacterium tuberculosis and protozoa such as Malaria parasites use the non-mevalonate (1-deoxy-d-xylulose-5-phosphate; DOXP) pathway for the phosphoantigenic biosynthesis (8). These antigens are presented to human Vγ9Vδ2 T-cells bound to the intracellular B30.2 domain of butyrophilin 3A1 (9). Antigens recognized by other human γδ T-cell subsets remain poorly defined. It has been suggested that Vδ1 T cells recognize MHC class I related molecules MICA, MICB, and ULBPs. In addition to the T-cell receptor, γδ T cells regularly express activating natural killer receptors, such as NKp30, NKp44, or NKG2D that recognize stress-inducible surface markers which are normally absent on healthy cells but expressed on malignant or infected cells (1, 10,11). With these independent, but synergic recognition modes in addition to the HLA-unrestricted antigen recognition, γδ T cells are considered to have great potential for clinical interventions.

WO2019104269A1 discloses a method of activating and expanding γδ T cells comprising isolating γδ T cells from a blood sample of a human subject, activating the isolated γδ T cells in the presence of an aminobisphosphonate, human recombinant interleukin 2 (IL-2), and human recombinant interleukin 15 (IL-15), and expanding the activated γδ T cells in the absence of an aminobisphosphonate and in the presence of human recombinant interleukin 2 (IL-2) and human recombinant interleukin 15 (IL-15). The method may further comprise transducing the activated γδ T cells with a recombinant viral vector. Such a viral recombinant vector may be an RD114TR pseudotyped lentiviral vector.

The prior art shows that γδ T cells are typically difficult to be transduced by viral vectors.

The use of chimeric antigen receptor (CAR)-expressing immune cells such αβ T cells re-directed to specifically recognize and eliminate malignant cells greatly increased the scope and potential of adoptive immunotherapy and is being assessed for a new standard of care in certain human malignancies. CARs are recombinant receptors that typically target surface molecules in a human leukocyte antigen (HLA)-independent manner. Generally, CARs comprise an extracellular antigen recognition moiety, often a single-chain variable fragment (scFv) derived from antibodies or a Fab fragment, linked to an extracellular spacer, a transmembrane domain and intracellular co-stimulatory and signaling domains. Therapies using CAR-engineered T cells, although sometimes efficacious, have a high potential for improvements, especially with regard to safety.

“Universal” CAR systems (or adapter CAR systems) that indirectly bind to target cells via soluble factors are described in the art. E.g. in WO2012082841A2, WO2013044225A1 and WO2016030414A1 tagged antibodies and tag-specific CAR are disclosed, wherein the tag may be either artificial (such as FITC) and potentially immunogenic or an endogenous molecule which may compete with the natural counterparts to the CAR binding.

Therefore, there is a need in the art for improved or alternative methods for efficient transduction of γδ T cells with retroviral vectors for introducing a nucleic acid encoding a transgene such as a CAR or TCR into said γδ T cells and optionally for the subsequent expansion of the genetically modified γδ T cells to high numbers which may allow in-vivo applications in subjects.

### BRIEF DESCRIPTION OF THE INVENTION

It is generally accepted that resting T cells cannot be easily transduced by the classical vesicular stomatitis virus G (VSVG-G) pseudotyped viral vectors due to the lack of the VSVG-receptor, overall in the absence of TCR stimulation or activation by CD3 or co-receptors like CD28. However, stimulation of T cells by TCR, CD3 or CD28 agonists alters the immune competence and final phenotype of γδ T cells for the use of these cells as immune effectors.

Unexpectedly the inventors have shown that the γδ T cells can be transduced with a high transduction efficiency by using a pseudotyped retroviral vector particle or virus-like particle thereof at low MOI that comprise a modified baboon endogenous retrovirus (BaEV) envelope glycoprotein as disclosed herein.

Additionally, the inventors surprisingly have found that γδ T cells, especially the Vγ9Vδ2 T cells expanded with amino bisphosphonates and low doses of cytokines such as of IL-2 and IL-15 exhibit an appropriate cell phenotype for adoptive cell-based therapies comprised mainly of effector memory and central memory cells.

In contrast to the common protocols for the expansion of primary αβ T cells that include co-culturing with human serum and other supporting cells as a source of favorable growth factors, the inventors have shown that the γδ T cells from these blood sources can be expanded in the absence of such supportive cells or factors in the presence of amino bisphosphonates and low doses of cytokines retaining the adequate cell phenotype.

Therefore, γδ T cells transduced with a pseudotyped retroviral vector as disclosed herein and expanded as disclosed herein may result in high cell numbers of genetically modified γδ T cells that exert the phenotype comprised mainly of effector memory and central memory cells and therefore being beneficial for adoptive cell-based therapies.

The processes of the invention allow the modification and expansion of γδ T cells in an allogeneic setting, i.e. the application of γδ T cells in a composition obtained by the methods as disclosed herein in an allogeneic treatment, for example in the treatment of cancer in a subject suffering from said cancer. The modified γδ T cells for such a treatment may express a chimeric antigen receptor or an exogenous TCR specific for an antigen expressed on the surface of cancer cell of said cancer.

Adapter CAR (adCAR) systems allow to “inactivate” immune cells that express the adCAR by withdrawal of the adapter. This leads to fewer side effects in a subject treated with an adapterCAR immune cell therapy if said immune cells are persistent in said subject. But the adapter may be administered again to said subject and again activate said immune cells expressing that adCAR, if required. The surprising finding that genetically modified γδ T cells that are transduced and expanded by the methods disclosed herein exert the phenotype comprised mainly of effector memory and central memory cells, i.e. they show persistence in a subject, makes them ideal candidates for CAR T cell immunotherapy based on the adapterCAR system. Said phenotype is manifested by the differential expression of the markers CD45RA and CD27 on the cell surface of the transduced and expanded γδ T cells.

Naïve CD45RA+CD27+γδ T cells have been reported to account for ˜15-30% of the circulating γδ T cell population in healthy human adults with a higher presence in lymph nodes. CD45RA−CD27+ central memory (TCM) γδ T cells represent 25-40% in peripheral blood with a marked inter-donor variability and a lower number in lymph nodes, while the CD45RA−CD27− effector memory (TEM) population is barely present in lymph nodes with a variable presence in peripheral blood (15-30%). The CD45RA+CD27− subset represent a fully differentiated subset with minimal number in blood and lymph nodes but enriched in inflammatory niches. Despite the high proliferative capacity, naïve γδ T cells lack most of the effector characteristics that memory and effector memory γδ T cells have (12, 13). Ideally, a good cellular product for adoptive cell therapies should pose good effector characteristics while keeping the proliferative attributes.

Therefore, a high percentage of CD45RA−CD27+ central memory (TCM) γδ T cells and CD45RA−CD27− effector memory (TEM) γδ T cells, i.e. a high percentage of CD45RA⁻γδ T cells, are beneficial for in-vivo applications. Therefore, generally, a composition of genetically modified γδ T cells transduced with the pseudotyped retroviral vector as disclosed herein and expanded as disclosed herein have beneficial in-vivo effects, wherein in said composition at least 75%, at least 80% or at least 90% of the γδ T cells are CD45RA⁻ γδ T cells.

In addition, in the context of adoptive cell therapies against cancer, the use of NK cells and γδ T cells with the same CAR target moieties offers a suitable platform for allogeneic and off-the-shelf medical interventions. Both cell types share different characteristics of innate effectors but also provide synergistic capacities to modulate the immune response against both solid and haematological malignancies. The BaEV transduction system as disclosed herein offers the opportunity to create a potent allogeneic product with the same CAR target in addition to the natural anti-tumour reactivity of γδ T cells and NK cells.

### BRIEF DESCRIPTION OF THE DRAWINGS

FIG. 1. Optimization of a method for the establishment of a highly effective transduction method. Different factors were tested in a custom Design of Experiment (DoE).

FIG. 2. Comparison of transduction efficiency by flow cytometry seven days after transduction with two pseudotypes: VSV-G and BaEV. The optimal conditions with two pseudotypes and two CAR constructs were further tested, and the proportion of cells expressing the CAR (A) as well as the concentration of CAR expressed on each cell (B) were compared, and thus at different MOI (C).

FIG. 3. Impact of transduction on the expansion of γδ T cells. Untransduced cells and cells transduced with CD19 CAR or CD33 CAR were compared.

FIG. 4. Impact of transduction on the phenotype of γδ T cells at day 14. Untransduced cells, the CAR negative population of transduced cells and the CAR positive population of transduced cells were compared. CM=central memory, EM=effector memory, EMRA=effector memory re-expressing CD45RA which are considered terminally differentiated.

FIG. 5. Impact of transduction on the activation of γδ T cell at day 14. The activation markers CD69 (A), CD56 (B), HLA-DR (C) and PD-1 (D) were measured. Untransduced cells, the CAR negative population of transduced cells and the CAR positive population of transduced cells were compared.

FIG. 6. Functional analysis of CD19 CAR (A) and CD33 CAR (B) γδ T cells against RS4,11 and OCI-AML3 WT and CD33 KO cell lines. γδ T cells showed an increased cytotoxicity when they were transduced with a CAR and were in presence of the CAR target. Cells expanded from three donors are compared (shown with the circle, square and triangle shapes).

FIG. 7. Activation and expansion of Vγ9Vδ2 T cells with Zoledronate and cytokines in the presence of serum. High expansion folds (A) and γδ T cell purity (B and C) were achieved in two weeks of expansion. These γδ T cells had a favorable phenotype (D) and activation profile throughout the expansion (E) and at the end (F).

FIG. 8. Expansion of Vγ9Vδ2 T cells in the presence (10%) and absence of human AB serum. Their expansion (A), cellular composition (B), phenotype (C) and activation profile (D) were compared.

FIG. 9. Functional analysis of γδ T cells against K562 and Raji cells. Untransduced γδ T cells showed high cytotoxicity (A and B), partly thanks to their degranulation (C) and secretion properties of different cytokines and cytotoxic granules (D).

FIG. 10. Number of γδ T cells and CAR+γδ T cells that could be obtained from 1×10⁹ PBMC.

FIG. 11. Expansion of γδ T cells with an αβTCR depletion step with the Prodigy. This additional step allows to further increase γδ T cell purity at the end of the expansion.

FIG. 12. Expansion and transduction process. Peripheral blood mononuclear cells (PBMC) are isolated from peripheral blood. The depletion of unwanted cell populations is possible before the activation and expansion of the γδ T cells. After a short amount of time, the cells are transduced and further expanded. They are then suitable cell products for cellular therapies.

FIG. 13. (A) Degranulation of CAR γδ T cells after expansion with and without serum, (B) Expression of IFNγ in CAR γδ T cells after expansion with and without serum, (C) Expression of IL-2 in CAR γδ T cells after expansion with and without serum.

### DETAILED DESCRIPTION OF THE INVENTION

In a first aspect, the present invention provides an in-vitro method for transferring (transducing) one or more nucleic acids sequences comprising one or more transgenes into γδ T cells with a pseudotyped retroviral vector particle or a virus-like particle thereof, comprising the steps
a) activation of γδ T cells of a sample that is a whole blood sample, a leukapheresis, a buffy coat or a PBMC sample, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample.

In another aspect, the present invention provides an in-vitro method for transferring one or more nucleic acids sequences comprising one or more transgenes into γδ T cells with a pseudotyped retroviral vector particle or a virus-like particle thereof, comprising the steps
a) removing αβ T cells from a sample that is a whole blood sample, a leukapheresis, a buffy coat or a PBMC sample, and
b) Activation of the γδ T cells of the sample of a), and
c) contacting said pseudotyped retroviral vector particle or virus-like particle thereof with said activated γδT cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample.

Said removing of αβ T cells from said sample may be performed by labeling the αβ T cells of said sample with an antibody or antigen binding fragment thereof specific for alpha beta T cell receptor (TCR), e.g. an antibody or antigen binding fragment thereof specific or the alpha chain of the TCR or the beta chain of the TCR.

In addition to removing αβ T cells form said sample other cells may be removed from said sample.

Said method, wherein said removing also comprises removing B cells and/or NK cells.

Said method, wherein said removing also comprises removing other cells but saves monocytes and granulocytes in the sample.

Said method, wherein said removing also comprises depletion of CD19 positive cells.

In some embodiments of the invention the method comprises between removing in step a) and activation in step b) an enrichment step.

Said method, wherein said enrichment step may be the enrichment of γδ T cells or the enrichment of CD45 positive cells.

Said methods, wherein said contacting of said pseudotyped retroviral vector particle or virus-like particle thereof with said activated γδ T cells is in the presence of a (retroviral) transduction enhancer.

Transduction enhancers for retroviral vectors are well-known in the art. The transduction enhancer may be e.g. RetroNectin® or VectoFusin-1®.

Said methods, wherein said pseudotyped retroviral vector particle or virus-like particle thereof comprises at least:
a chimeric envelope glycoprotein which comprises or consists in a fusion of the transmembrane and extracellular domain of a baboon endogenous retrovirus (BaEV) envelope glycoprotein and the cytoplasmic tail domain of a murine leukemia virus (MLV) envelope glycoprotein; or
a modified BaEV envelope glycoprotein wherein the cytoplasmic tail domain is devoid of the fusion inhibitory R peptide.

Said retroviral vector may be a lentiviral vector.

Said methods, wherein said activation of γδ T cells may be performed by adding to said γδ T cells an aminobisphosphonate together with one or more cytokine(s) selected from the group consisting of IL-1b, IL2, IL-7, IL-15, IL-18 and IL-21.

Said methods, wherein said activation of γδ T cells may be performed preferentially by adding to said γδ T cells an aminobisphosphonate together with IL2 and IL-15.

In one embodiment of the invention aminobisphosphonate may be zoledronate, pamidronate, ibandronate, incadronate, or a salt thereof and/or a hydrate thereof.

Said methods, wherein in said activation said aminobisphosphonate may be zoledronic acid or pamidronate.

Said methods, wherein in said activation said zoledronic acid may be at a concentration of 0.5 mM to 20 mM, 1 mM to 10 mM, 2 mM to 8 mM, 4 mM to 6 mM or 5 mM.

Said methods, wherein in said activation said zoledronic acid may be at a concentration of 0.5 mM to 20 mM, 1 mM to 10 mM, 2 mM to 8 mM, 4 mM to 6 mM or 5 mM, said IL-2 may be at a concentration of 10 IU/ml to 1000 IU/ml, 20 IU/ml to 800 IU/ml, 50 IU/ml to 701 U/ml to 600 IU/ml or 100 IU/ml and said IL-15 may be at a concentration of 10 IU/ml to 1000 IU/ml, 20 IU/ml to 800 IU/ml, 50 IU/ml to 70 IU/ml to 600 IU/ml or 100 IU/ml.

Said methods, wherein in said activation said zoledronic acid may at a concentration of 1 mM to 10 mM, said IL-2 may be at a concentration of 10 IU/ml to 1000 IU/ml, and said IL-15 may be at a concentration of 10 IU/ml to 1000 IU/ml.

Said methods, wherein said pseudotyped retroviral vector particle or virus-like particle thereof may be in a concentration of 0.01 MOI to 100 MOI, 0.01 MOI to 10 MOI, or 0.1 MOI to 10 MOI or 0.1 MOI to 1 MOI.

Said methods, wherein said pseudotyped retroviral vector particle or virus-like particle thereof preferentially may be in a concentration of 0.1 MOI to 1 MOI.

Said methods, wherein said activation of γδ T cells is in the absence of TCR stimulation or activation by CD3 or co-receptors like CD28.

Said methods, wherein said activation of γδ T cells is in the absence of anti-CD3 and anti-CD28 antibodies or antigen binding fragments thereof.

Said methods, wherein said transduction, i.e. said contacting said pseudotyped retroviral vector particle or virus-like particle thereof with said activated γδ T cells, may be at day 3 after activation of the γδ T cells.

Said method, wherein said methods additionally comprises the step of expanding said genetically modified γδ T cells.

Said methods, wherein said expansion comprises:
expanding the activated γδ T cells in the absence of an aminobisphosphonate and in the presence of one or more cytokines selected from the group consisting of IL-1b, IL-2, IL-7, IL-15, IL-18 and IL-21.

Said methods, wherein said expansion comprises:
expanding the activated γδ T cells in the absence of an aminobisphosphonate and in
the presence of IL-2 and IL-15.

Said method, wherein said expansion is in the presence of IL-2 and IL-15.

Said methods, wherein said expansion is in the absence of human serum.

Said methods, wherein said expansion is in the absence of feeder cells.

Said methods, wherein said expansion is in the absence of feeder cells and in the presence of human serum.

Said methods, wherein said expansion is in the absence of human serum and of feeder cells.

Said methods, wherein the genetically modified γδ T cells may expand in the absence of human serum and of feeder cells at least 300-fold, at least 400-fold, at least 500-fold during said step of expansion.

Said methods, wherein the genetically modified γδ T cells may expand in the absence of human serum and of feeder cells at least 300-fold, at least 400-fold, at least 500-fold at day 14 of expansion.

Said methods, wherein the genetically modified γδ T cells may expand in the presence of human serum.

Said methods, wherein the genetically modified γδ T cells may expand in the presence of human serum at least 300-fold, at least 400-fold, at least 500-fold during said step of expansion.

Said methods, wherein the genetically modified γδ T cells may expand in the presence of human serum at least 300-fold, at least 400-fold, at least 500-fold at day 14 of expansion.

Said methods, wherein said expansion comprises:
expanding the activated γδ T cells in the absence of an aminobisphosphonate (i.e. no novel addition of an aminobisphosphonate after said activation step) and of human serum and of feeder cells in the presence of IL-2 and IL-15.

Said methods, wherein in said expansion said IL-2 is at a concentration of 10 IU/ml to 1000 IU/ml, 20 IU/ml to 800 IU/ml, 50 IU/ml to 70 IU/ml to 600 IU/ml or 100 IU/ml and said IL-15 is at a concentration of 10 IU/ml to 1000 IU/ml, 20 IU/ml to 800 IN/ml, 50 IU/ml to 70 IU/ml to 600 IU/ml or 100 IU/ml.

Said methods, wherein the γδ T cells that are expanded in the expansion step are Vγ9Vδ2 T-cells.

Said method, wherein at least 75%, at least 80% or at least 90% of the transduced and expanded γδ T cells are CD45RA⁻γδ T cells, i.e. CD45RA⁻CD27+ and/or CD45RA⁻CD27⁻γδ T cells.

Said method of transduction and expansion of γδ T cells as disclosed herein, thereby achieving at least 75%, at least 80% or at least 90% of the transduced and expanded γδ T cells being CD45RA⁻γδ T cells, i.e. CD45RA⁻CD27+ and/or CD45RA⁻CD27⁻γδ T cells.

The cultivation of the cells for activation and/or expansion may be in basal medium for animal or human cells supplemented with the substances for specific activation of γδ T cells and/or for the expansion of the γδ T cells as disclosed herein. An example for such a medium and used herein is TexMACS™ (Miltenyi Biotec).

In one embodiment of the invention, a duration of activation in the methods as disclosed herein is from 1 day to about 5 days, from 1 day to about 4 days, from 1 day to about 3 days, or from 1 day to about 2 days.

Said methods, wherein said activation is from day 0 to day 3, from day 0 to day 2 or from day 0 to day 1 of the method.

In one embodiment of the invention, a duration of expansion in the methods as disclosed herein is about 7 days, about 10 days, about 12 days, about 14 days, about 15 days, about 16 days.

Said methods, wherein said methods are performed in a closed system in an automated manner. In one embodiment of the invention the method comprises an automated method in a closed system comprising:
a) preparation of a sample that is a PBMC sample by centrifugation
b) magnetic removal of the αβ T cells of the sample of a)
c) activation of the γδ T cells of sample b)
d) transducing the activated γδ T cells with a pseudotyped retroviral vector particle, wherein said pseudotyped retroviral vector particle comprises a modified baboon endogenous retrovirus (BaEV) envelope glycoprotein that is able of binding to and fusing with a hematopoietic cell membrane, thereby genetically modifying said γδ T cells,
e) expanding said genetically modified γδ T cells,
thereby generating a population of genetically modified γδ T cells.

Said method in said closed system, wherein after step b) or concomitantly with step b) (and before step c)) the method comprises the additional step of depletion of CD19 positive cells and/or CD34 positive cells.

Said method in said closed system, wherein the activation of the γδ T cells is with zoledronic acid, IL-2 and said IL-15.

Said method in said closed system, wherein the transduction is at low MOI, e.g. 0.1 MOI to 1 MOI.

Said method in said closed system, wherein the expansion of said genetically modified γδ T cells is in the absence of an aminobisphosphonate and in the presence of IL-2 and IL-15.

Said expanding of the activated γδ T cells in the absence of an aminobisphosphonate (i.e. no novel addition of an aminobisphosphonate after said activation step) and in the presence of IL-2 and IL-15 may be without human serum and feeder cells.

Alternatively, said expanding of the activated γδ T cells in the absence of an aminobisphosphonate (i.e. no novel addition of an aminobisphosphonate after said activation step) and in the presence of IL-2 and IL-15 may be with human serum (and without feeder cells).

Said methods, wherein said one or more transgene is a chimeric antigen receptor (CAR), a chemokine receptor and/or an exogenous T cell receptor (TCR).

In another aspect the present invention provides an in-vitro method for transferring one or more nucleic acids sequences comprising one or more transgenes into γδ T cells with a pseudotyped retroviral vector particle or a virus-like particle thereof, comprising the steps
a) Activation of said γδ T cells, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample.

In another aspect the present invention provides an in-vitro method for transferring one or more nucleic acids sequences comprising one or more transgenes into isolated γδ T cells with a pseudotyped retroviral vector particle or a virus-like particle thereof, comprising the steps
a) Activation of isolated γδ T cells, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample.

Said method, wherein said isolated γδ T cells are achieved by enrichment of γδ T cells from a sample that is a whole blood sample, a leukapheresis, a buffy coat or a PBMC sample.

Said enrichment of γδ T cells from said sample may be performed by labeling the γδ T cells of said sample with an antibody or antigen binding fragment thereof specific for gamma delta T cell receptor (TCR), e.g. an antibody or antigen binding fragment thereof specific or the gamma chain of the TCR or the delta chain of the TCR.

Said activation may be by addition of feeder cells or pyrophosphate metabolites such as isopentenyl pyrophosphate (IPP), hydroxy-methyl-butyl pyrophosphate (HMBPP) or bromohydrin pyrophosphate (BrHPP); monoclonal antibodies (MAbs) directed against the γδ TCRs or against co-stimulatory ligands expressed on γδ cells, such as NKG2D, CD70, JAML, DNAX accessory molecule-1 (DNAM-1), ICOS, CD27, CD137, CD30, CD270, CD150, CD122, and CD28. Co-stimulatory agents can be antibodies specific to unique epitopes on CD2 and CD3 molecules. CD2 and CD3 can have different conformation structures when expressed on αβ or γδ T-cells. Specific antibodies to CD3 and CD2 can lead to distinct activation of γδ T cells.

In another aspect the present invention provides a composition comprising γδ T cells, wherein at least 75%, at least 80% or at least 90% of said γδ T cells are CD45RA⁻ γδ T cells.

Said composition, wherein said γδ T cells are genetically modified γδ T cells.

Said genetically modified CD45RA⁻ γδ T cells may express a transgene such as a CAR.

Said composition, wherein said composition comprises γδ T cells transduced with a pseudotyped retroviral vector particle comprising one or more transgenes, wherein at least 75%, at least 80% or at least 90% of said γδ T cells are CD45RA⁻ γδ T cells, and wherein said pseudotyped retroviral vector particle comprises a modified baboon endogenous retrovirus (BaEV) envelope glycoprotein that is able of binding to and fusing with a hematopoietic cell membrane.

Said composition, wherein said CD45RA⁻ γδ T cells are obtained by an in-vitro method comprising expanding activated γδ T cells in the absence of an aminobisphosphonate and in the presence of one or more cytokines selected from the group consisting of IL-1b, IL-2, IL-7, IL-15, IL-18 and IL-21.

Said composition, wherein said genetically modified CD45RA⁻ γδ T cells are obtained by an in-vitro method comprising
a) activation of γδ T cells, and
b) contacting said activated γδ T cells with a viral vector such as a retroviral vector comprising one or more transgenes, thereby transducing said γδ T cells
c) expanding said transduced γδ T cells in the absence of an aminobisphosphonate and in the presence of one or more cytokines selected from the group consisting of IL-1b, IL-2, IL-7, IL-15, IL-18 and IL-21.

Said composition, wherein said CD45RA⁻ γδ T cells are genetically modified by transduction with a pseudotyped retroviral vector particle or virus-like particle thereof that comprises a modified baboon endogenous retrovirus (BaEV) envelope glycoprotein that is able of binding to and fusing with a hematopoietic cell membrane.

Said composition, wherein said genetically modified CD45RA⁻ γδ T cells are obtained by an in-vitro method for transferring (transducing) one or more nucleic acids sequences comprising one or more transgenes into γδ T cells with a pseudotyped retroviral vector particle or a virus-like particle thereof, comprising the steps
I)
a) activation of γδ T cells of a sample that is a whole blood sample, a leukapheresis, a buffy coat or a PBMC sample, wherein said activation of γδ T cells is performed by adding to said γδ T cells an aminobisphosphonate together with one or more cytokine(s) selected from the group consisting of IL-1b, IL-2, IL-7, IL-15, IL-18 and IL-21, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
c) expanding the activated γδ T cells in the absence of an aminobisphosphonate and in the presence of one or more cytokines selected from the group consisting of IL-1b, IL-2, IL-7, IL-15, IL-18 and IL-21,
or
II)
a) removing αβ T cells form a sample that is a whole blood sample, a leukapheresis, a buffy coat or a PBMC sample, and
b) Activation of the γδ T cells of the sample of a), wherein said activation of γδ T cells is performed by adding to said γδ T cells an aminobisphosphonate together with one or more cytokine(s) selected from the group consisting of IL-1b, IL2, IL-7, IL-15, IL-18 and IL-21 and
c) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
d) expanding the activated γδ T cells in the absence of an aminobisphosphonate and in the presence of one or more cytokines selected from the group consisting of IL-1b, IL-2, IL-7, IL-15, IL-18 and IL-21.

Said one or more transgenes may be a CAR. Said CAR may be specific for an antigen expressed on the surface of a target cell such as a cancer cell or may be a soluble antigen.

In another aspect the present invention provides a composition (or a kit or a combination) comprising
i) a composition comprising γδ T cells expressing a CAR comprising
a) an antigen binding domain specific for a tag of a tagged polypeptide
b) a transmembrane domain
c) an intracellular signaling domain,
ii) said tagged polypeptide, wherein said polypeptide binds specifically to an antigen expressed on the surface of a target cell or binds specifically to a soluble antigen.

Said composition, wherein in said composition comprising γδ T cells at least 75%, at least 80%,
at least 90% of the γδ T cells are CD45RA⁻ γδ T cells.

Said composition as disclosed herein, wherein said polypeptide of said tagged polypeptide may be or may comprise an antibody or antigen binding fragment thereof.

Said tag of said tagged polypeptide may be a hapten.

Said tag of said tagged polypeptide may be dextran, biotin, fluorescein isothiocyanate (FITC), phycoerythrin (PE), peptides such as c-Myc-tag, Strep-Tag, Flag-Tag, Polyhistidine-tag or proteins such as streptavidin.

Said intracellular signaling domain may comprise at least one primary cytoplasmic signaling domain comprising an immunoreceptor tyrosine-based activation motif (ITAM) and/or at least one co-stimulatory signaling domain.

Said primary cytoplasmic signaling domain of said first CAR may be CD3zeta.

Said at least one co-stimulatory domain of said first CAR, may be selected from the group consisting of ICOS, CD154, CD5, CD2, CD46, HVEM, CD8, CD97, TNFRSF18, CD30, SLAM, DAP10, CD64, CD16, CD89, MyD88, KIR-2DS, KIR-3DS, NKp30, NKp44, NKp46, NKG2D, ICAM, CD27, OX40, 4-1BB, and CD28.

Said γδ T cells may be Vγ9Vδ2 T-cells.

Said target cell may be a cancer cell, a cell associated with an autoimmune disease or associated with an infectious disease.

Said soluble antigen may be a soluble antigen of a tumor microenvironment (TME), a soluble antigen specifically associated with an autoimmune disease, or a soluble antigen specifically associated with an infectious disease.

The disease may be a cancer, e.g. a solid cancer or may be a lymphoma or a hematological malignancy.

Said solid cancer (tumor) may be adrenal cancer, anal cancer, bile duct cancer, bladder cancer, bone cancer, brain/CNS tumors in children or adults, breast cancer, cervical cancer, colon/rectum cancer, endometrial cancer, esophagus cancer, ewing family of tumors, eye cancer, gallbladder cancer, gastrointestinal carcinoid tumors, gastrointestinal stromal tumor (GIST), gestation trophoblastic disease, hodgkin disease, kaposi sarcoma, kidney cancer, laryngeal and hypopharyngeal cancer, leukemia, acute lymphocytic leukemia, acute myeloid leukemia, chronic lymphocytic leukemia, chronic myeloid leukemia, chronic myelomonocytic leukemia, liver cancer, lung cancer, non-small cell lung cancer, small cell lung cancer, lung carcinoid tumor, lymphoma, malignant mesothelioma, multiple myeloma, myelodysplastic syndrome, nasal cavity and paranasal sinum cancer, nasopharyngeal cancer, neuroblastoma, non-hodgkin lymphoma, oral cavity or oropharyngeal cancer, osteosarcoma, ovarian cancer, pancreatic cancer, penile cancer, prostate cancer, rhabdomyosarcoma, skin cancer, melanoma, merkel cell skin cancer, small intestine cancer, stomach cancer, testicular cancer, thyroid cancer, uterine sarcoma, vaginal cancer, vulvar cancer, or nephroblastoma.

Autoimmune diseases are a condition arising from autoimmunity or disbalance in the immune homeostasis resulting in pathologies that can affect multiple different organ systems. Examples include Behcet's disease, Juvenile idiopathic arthritis, Type 1 diabetes, Rheumatoid arthritis, Wegener Granulomatosis, Systemic lupus erythematosus, Systemic sclerosis, Crohn's disease, Graves' disease, Hashimoto thyroiditis, Goodpasture syndrome, Primary biliary cholangitis, Myasthenia gravis, Dermato polymyositis, Vasculitis, Mixed connective tissue disease, Scleroderma, Multiple sclerosis, Psoriasis, Ulcerative colitis and Uvetis.

Therefore, said autoimmune disease may be e.g. Behcet's disease, Juvenile idiopathic arthritis, Type 1 diabetes, Rheumatoid arthritis, Wegener Granulomatosis, Systemic lupus erythematosus, Systemic sclerosis, Crohn's disease, Graves' disease, Hashimoto thyroiditis, Goodpasture syndrome, Primary biliary cholangitis, Myasthenia gravis, Dermato polymyositis, Vasculitis, Mixed connective tissue disease, Scleroderma, Multiple sclerosis, Psoriasis, Ulcerative colitis or Uvetis.

Infection is the invasion of an organism's body tissues by disease-causing agents, their multiplication, and the reaction of host tissues to the infectious agents and the toxins they produce. Infections are caused by infectious agents (pathogens) including: viruses, bacteria, fungi and parasites. Said infection may be an acute or a chronic infection.

In one embodiment of the invention the composition (or a kit or a combination) is a composition (or a kit or a combination) comprising
i) a composition comprising γδ T cells expressing a CAR comprising
a) an antigen binding domain specific for a tag of a tagged polypeptide
b) a transmembrane domain
c) an intracellular signaling domain,
ii) said tagged polypeptide, wherein said polypeptide binds specifically to an antigen expressed on the surface of a target cell or binds specifically to a soluble antigen;
wherein said composition of γδ T cells expressing said CAR is obtained by the in-vitro method comprising the steps
I)
a) activation of γδ T cells of a sample that is a whole blood sample, a leukapheresis, a buffy coat or a PBMC sample, wherein said activation of γδ T cells is performed by adding to said γδ T cells an aminobisphosphonate together with one or more cytokine(s) selected from the group consisting of IL-1b, IL2, IL-7, IL-15, IL-18 and IL-21, and
b) contacting a viral vector particle or virus-like particle thereof comprising a nucleic acid sequence encoding said CAR with said activated γδ T cells,
c) expanding the activated γδ T cells of step b) in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR,
or
II)
a) removing αβ T cells form a sample that is a whole blood sample, a leukapheresis, a buffy coat or a PBMC sample, and
b) activation of the γδ T cells of the sample of a), wherein said activation of γδ T cells is performed by adding to said γδ T cells an aminobisphosphonate together with one or more cytokine(s) selected from the group consisting of IL-1b, IL2, IL-7, IL-15, IL-18 and IL-21, and
c) contacting a viral vector particle or virus-like particle thereof comprising a nucleic acid sequence encoding said CAR with said activated γδ T cells,
d) expanding the activated γδ T cells of step c) in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR.

Said viral vector may be a retroviral vector such as a lentiviral vector. Said retroviral vector may be a pseudotyped retroviral vector.

For example, the pseudotyped retroviral vectors may be lentiviral vectors pseudotyped with envelope glycoproteins (GPs) from the vesicular stomatitis virus (VSV-G), the modified feline endogenous retrovirus (RD1 14TR), the modified gibbon ape leukemia virus (GALVTR) or from the BaEV envelope glycoprotein as disclosed herein.

Alternatively, said retroviral vector may be a gamma retroviral vector or an adeno-associated viral vector.

In one embodiment of the invention the composition (or a kit or a combination) is a composition (or a kit a combination) comprising
i) a composition comprising γδ T cells expressing a CAR comprising
a) an antigen binding domain specific for a tag of a tagged polypeptide
b) a transmembrane domain
c) an intracellular signaling domain,
ii) said tagged polypeptide, wherein said polypeptide binds specifically to an antigen expressed on the surface of a target cell or binds specifically to a soluble antigen;
wherein said composition of γδ T cells expressing said CAR is obtained by the in-vitro method for transferring one or more nucleic acids sequences comprising said CAR into γδ T cells with a pseudotyped retroviral vector particle or a virus-like particle thereof, the method comprising the steps
I)
a) activation of γδ T cells of a sample that is a whole blood sample, a leukapheresis, a buffy coat or a PBMC sample, wherein said activation of γδ T cells is performed by adding to said γδ T cells an aminobisphosphonate together with one or more cytokine(s) selected from the group consisting of IL1b, IL2, IL-7, IL-15, IL-18 and IL-21, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
c) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR,
or
II)
a) removing αβ T cells form a sample that is a whole blood sample, a leukapheresis, a buffy coat or a PBMC sample, and
b) Activation of the γδ T cells of the sample of a), wherein said activation of γδ T cells is performed by adding to said γδ T cells an aminobisphosphonate together with one or more cytokine(s) selected from the group consisting of IL-1b, IL2, IL-7, IL-15, IL-18 and IL-21 and
c) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
d) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR.

Said compositions as disclosed herein for use in treatment of a disease (in a subject) such as cancer, an infectious disease or an autoimmune disease.

In a further aspect the present invention provides a pharmaceutical composition comprising a therapeutically effective amount of genetically modified γδ T cells obtained by the methods as disclosed herein, and optionally a pharmaceutically acceptable carrier.

Pharmaceutically acceptable carriers, diluents or excipients may comprise buffers such as neutral buffered saline, phosphate buffered saline and the like; isotonic saline solutions, carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In a further aspect the present invention provides a composition comprising
a) genetically modified γδ T cells transduced with a pseudotyped retroviral vector particle or a virus-like particle thereof comprising one or more transgenes,
b) genetically modified NK cells transduced with a pseudotyped retroviral vector particle or a virus-like particle thereof comprising one or more transgenes,
wherein said pseudotyped retroviral vector particle or virus-like particle thereof comprises a modified baboon endogenous retrovirus (BaEV) envelope glycoprotein that is able of binding to and fusing with a hematopoietic cell membrane.

Said composition, wherein at least 75%, at least 80% or at least 90% of said γδ T cells are CD45RA⁻ γδ T cells.

Said composition, wherein said genetically modified γδ T cells are obtained by an in-vitro method for transferring (transducing) one or more nucleic acids sequences comprising one or more transgenes into γδ T cells with a pseudotyped retroviral vector particle or a virus-like particle thereof, comprising the steps
I)
a) activation of γδ T cells of a sample that is a whole blood sample, a leukapheresis, a buffy coat or a PBMC sample, wherein said activation of γδ T cells is performed by adding to said γδ T cells an aminobisphosphonate together with one or more cytokine(s) selected from the group consisting of IL-1b, IL2, IL-7, IL-15, IL-18 and IL-21, and
b) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
c) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition, wherein at least 75%, at least 80% or at least 90% of said γδ T cells are CD45RA⁻ γδ T cells,
or
II)
a) removing αβ T cells form a sample that is a whole blood sample, a leukapheresis, a buffy coat or a PBMC sample, and
b) Activation of the γδ T cells of the sample of a), wherein said activation of γδ T cells is performed by adding to said γδ T cells an aminobisphosphonate together with one or more cytokine(s) selected from the group consisting of IL-1b, IL2, IL-7, IL-15, IL-18 and IL-21 and
c) contacting said pseudotyped retroviral vector particle or virus-like particle thereof with said activated γδ T cells,
wherein said pseudotyped retroviral vector particle or virus-like particle thereof comprises a modified baboon endogenous retrovirus (BaEV) envelope glycoprotein that is able of binding to and fusing with a hematopoietic cell membrane, thereby genetically modifying said γδ T cells of said sample,
d) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition wherein at least 75%, at least 80% or at least 90% of said γδ T cells are CD45RA⁻γδ T cells.

NK cell transduction with said BaEV pseudotyped retroviral vector particle or virus-like particle are well-known in the art and described e.g. in WO2019121945A1.

Said composition, wherein said genetically modified NK cells are obtained by an in-vitro method for transferring one or more nucleic acids into activated NK cells with a pseudotyped retroviral vector particle or a virus-like particle thereof, comprising the steps
a) Activation of NK cells, and
b) Addition of said pseudotyped retroviral vector particle or virus-like particle thereof to said activated NK cells,
wherein said pseudotyped retroviral vector particle or virus-like particle thereof comprises a modified baboon endogenous retrovirus (BaEV) envelope glycoprotein that is able of binding to and fusing with a hematopoietic cell membrane.

Said activation of said NK cells may be achieved by the addition of at least one cytokine or feeder cells or membrane particles of feeder cells or a with an NK cell activation reagent to said NK cells. Said at least one cytokine may be IL-2 and/or IL-15. Said activation of NK cells may be achieved by the addition of a combination of cytokines comprising at least one cytokine that activates NK cells and a IL-1 family cytokine. Said combination of cytokines may be IL2 and/or IL-15 and a IL-1 family cytokine. Said IL-1 family cytokine may be IL-18, IL-33 or IL-1beta.

Said composition, wherein said one or more transgenes is a TCR.

Said composition, wherein said one or more transgenes is a CAR and said genetically modified T cells and said genetically modified NK cells express said CAR.

The CAR may be identical in both, the genetically modified γδ T cells and the genetically modified NK cells. This allows the simultaneous transduction of said γδ T cells and NK cells with said pseudotyped retroviral vector particle or virus-like particle thereof that comprises a modified baboon endogenous retrovirus (BaEV) envelope glycoprotein that is able of binding to and fusing with a hematopoietic cell membrane.

Said CAR may be specific for an antigen expressed on the surface of a target cell such as a cancer cell or may be specific for a soluble antigen (e.g. naturally occurring in a tumor microenvironment). Said CAR may be an adapterCAR as also described herein.

Alternatively, the CARs may be different in said γδ T cells and said NK cells.

In another aspect the present invention provides an in-vivo method for treatment of a disease in a subject, the method comprising,
i) administering to a subject in need thereof a composition comprising a therapeutically effective amount of CD45RA⁻γδ T cells expressing a transgene such as a CAR or a TCR.

In another aspect the present invention provides an in-vivo method for treatment of a disease in a subject, the method comprising,
i) administering to a subject in need thereof a composition comprising γδ T cells expressing a CAR comprising
a) an antigen binding domain specific for a tag of a tagged polypeptide
b) a transmembrane domain
c) an intracellular signaling domain, and
ii) administering to said subject said tagged polypeptide, wherein said polypeptide binds specifically to an antigen expressed on the surface of a target cell or binds specifically to a soluble antigen;
wherein said composition of γδ T cells expressing said CAR is obtained by the in-vitro method for transferring one or more nucleic acids sequences comprising said CAR into γδ T cells with a viral vector particle or a virus-like particle thereof, the method comprising the steps
I)
a) activation of γδ T cells of a sample that is a whole blood sample, a leukapheresis, a buffy coat or a PBMC sample, wherein said activation of γδ T cells is performed by adding to said γδ T cells aminobisphosphonate together with one or more cytokine(s) selected from the group consisting of IL-1b, IL2, IL-7, IL-15, IL-18 and IL-21, and
b) contacting said viral vector particle or virus-like particle thereof with said activated TS T cells,
c) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR,
or
II)
a) removing αβ T cells form a sample that is a whole blood sample, a leukapheresis, a buffy coat or a PBMC sample, and
b) Activation of the γδ T cells of the sample of a), wherein said activation of γδ T cells is performed by adding to said γδ T cells aminobisphosphonate together with one or more cytokine(s) selected from the group consisting of IL-1b, IL2, IL-7, IL-15, IL-18 and IL-21 and
c) contacting said viral vector particle or virus-like particle thereof with said activated TS T cells,
d) expanding the activated γδ T cells in the absence of an aminobisphosphonate and in the presence of one or more cytokines selected from the group consisting of IL-1b, IL-2, IL-7, IL-15, IL-18 and IL-21, thereby generating said composition comprising said γδ T cells expressing said CAR.

Said in-vivo method, wherein said viral vector may be a retroviral vector.

Said in-vivo method, wherein said retroviral vector may be a pseudotyped retroviral vector particle comprising a modified baboon endogenous retrovirus (BaEV) envelope glycoprotein that is able of binding to and fusing with a hematopoietic cell membrane.

Said in-vivo method, wherein said administration of said tagged polypeptide is repeated at least once, and wherein between said repeat is a period of at least 2 weeks, at least 1 month, at least 2 months, at least 3 months, at least 4 months, at least 5 months, at least 6 months, at least 7 months, at least 8 months, at least 9 months, at least 10 months, at least 11 months, or at least 12 months.

Said in-vivo method, wherein in addition to said administration of said tagged polypeptide also the administration of said composition comprising said γδ T cells may be repeated at least once, and wherein between said repeat is a period of at least 2 weeks, at least 1 month, at least 2 months, at least 3 months, at least 4 months, at least 5 months, at least 6 months, at least 7 months, at least 8 months, at least 9 months, at least 10 months, at least 11 months, or at least 12 months.

### Embodiments

In one embodiment of the invention the generation of γδ T cells expressing a CAR as disclosed herein are for use in treatment of a disease associated with a target cell of a subject suffering from said disease, the disease may be e.g. cancer and the target cell a cancerous cell. γδ T cells of a subject may be collected from a subject. The subject may e.g. suffer from said cancer or may be a healthy subject. These γδ T cells are genetically modified in-vitro to express a CAR as disclosed herein. In a cellular therapy these engineered γδ T cells may be infused to a recipient in need thereof. These cells may be a pharmaceutical composition (said cell plus pharmaceutical acceptable carrier). The infused cells may be e.g. able to kill (or at least stop growth of) cancerous cells in the recipient. The recipient may be the same subject from which the cells was obtained (autologous cell therapy) or may be from another subject of the same species (allogeneic cell therapy).

The γδ T cells engineered to express a CAR generated by the methods as disclosed herein may be administered either alone, or as a pharmaceutical composition in combination with diluents and/or with other components such as IL-2 or other cytokines or cell populations. Briefly, pharmaceutical compositions may comprise a cell population of genetically modified cells generated by the methods as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants; and preservatives.

Preferentially, the compositions comprising γδ T cells expressing a CAR are formulated for intravenous administration. The administration of cell compositions to the subject may be carried out in any convenient manner known in the art.

Pharmaceutical compositions may be administered in a manner appropriate to the disease to be treated. Appropriate dosages may be determined by clinical trials. But the quantity and frequency of administration will also be determined and influenced by such factors as the condition of the patient, and the type and severity of the patient's disease.

A pharmaceutical composition comprising the γδ T cells generated by the methods as disclosed herein may be administered at a dosage of 10⁴ to 10⁹ cells/kg body weight, preferably 10⁵ to 10⁶ cells/kg body weight. The cell compositions may also be administered several times at these dosages. The compositions of cells may be injected e.g. directly into a tumor, lymph node, or site of infection.

The genetically engineered γδ T cells may be activated and expanded to therapeutic effective amounts as disclosed herein.

The modified γδ T cells generated by the methods of the invention may be used in combination with e.g. chemotherapy, radiation, immunosuppressive agents, antibodies or antibody therapies. All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs. As used herein the term “comprising” or “comprises” is used in reference to compositions, methods, and respective component(s) thereof, that are essential to the method or composition, yet open to the inclusion of unspecified elements, whether essential or not.

Retroviridae is virus family with a single-stranded, diploid, positive-sense RNA genome that is reverse-transcribed into a DNA intermediate that is then incorporated into the host cell genome. Retroviridae-derived viruses are enveloped particles with a diameter of 80-120 nm.

(Retro-/lenti-/gammaretro-) viral vectors are replication-deficient viral particles that are derived from the corresponding virus family. They contain Gag and Pol proteins, a single-stranded RNA genome and are usually pseudotyped with heterologous envelope proteins derived from other viruses, e.g. with a baboon endogenous retrovirus (BaEV) envelope glycoprotein as disclosed herein. The RNA genome of said viral vectors do not contain any viral gene to produce viral progeny, but psi elements and LTRs that are required for efficient packing and reverse transcription in DNA. The DNA intermediate may contain a gene of interest under the control of a suitable promoter, for example, the CMV promoter and the gene of interest is expressed upon integration of said DNA into the genome of the host cell. The process of entering the host cell, delivering the RNA genome, integration and expression of the gene of interest is called transduction. The minimal requirements of a gammaretrovirus or lentivirus based viral vector has been well-described in the art.

In addition, integrase-deficient retroviral vectors (ID-RVs) have been developed that cannot integrate the retroviral vector genome in the host cell genome. ID-RVs are derived from conventional retroviral vectors but contain no or a mutated form of the retroviral integrase. Upon entry into the host cell, the retroviral vector genome is reverse-transcribed in the cytoplasm, delivered into the nucleus, but not stably integrated into the host cell genome. ID-RVs are a useful tools to express the gene of interest transiently. The definition of retroviral vectors and transduction also extents the integration-deficient retroviral vectors and its application.

Lentivirus is a genus of Retroviridae that cause chronic and deadly diseases characterized by long incubation periods, in the human and other mammalian species. The best known lentivirus is the Human Immunodeficiency Virus HIV which can efficiently infect nondividing cells, so lentiviral-derived retroviral vectors are one of the most efficient methods of gene delivery. Gammaretroviridae is a genus of the Retroviridae family. Representative species are the murine leukemia virus and the feline leukemia virus.

Virus-like particles (VLPs) resemble viral particles, but are not infecting or transducing because they contain no viral genetic material encoding for the proteins of the virus-like particle. In particular, VLPs in the context of retroviral vectors do not contain psi positive nucleic acid molecules. Some virus-like particles may contain nucleic acid distinct from their genome. The expression of viral structural proteins, such as envelope or capsid, can result in the assembly of virus like particles (VLPs). Like for retroviral vectors VLPs can also be pseudotyped using the same envelope constructs as for retroviral vectors. VLPs may be used to deliver proteins but also nucleic acids to the cytoplasm of target cells. In particular, VLPs are useful as vaccines.

The term “VLP uptake” as used herein refers to the binding of a VLP to the target cell membrane, thereby releasing nucleic acid molecules, proteins or peptides into the target cell.

The term “activation” as used herein refers to inducing physiological changes with a cell that increase target cell function, proliferation and/or differentiation.

The term “pseudotyping” or “pseudotyped” as used herein refers to a vector particle bearing envelope glycoproteins derived from other viruses having envelopes. The host range of the lentiviral vectors or vector particles of the present invention can thus be expanded or altered depending on the type of cell surface receptor used by the glycoprotein.

To generate retroviral vectors the gag, pol and env proteins needed to assemble the vector particle are provided in trans by means of a packaging cell line, for example, HEK-293T. This is usually accomplished by transfection of the packaging cell line with one or more plasmids containing the gag, pol and env genes. For the generation of pseudotyped vectors, the env gene, originally derived from the same retrovirus as the gag and pol genes and as the RNA molecule or expression vector, is exchanged for the envelope protein(s) of a different enveloped virus.

The Baboon endogenous retrovirus or BaEV is a type C retrovirus present in multiple proviral copies in the DNA of baboons. In WO2013045639A1 the wild-type BaEV envelope glycoprotein (non-modified BaEV envelope glycoprotein) and BaEV envelope glycoproteins having defined mutations (modifications) that were incorporated at a higher level on the lentiviral surface than the wild-type BaEV glycoprotein are described in detail.

The term “BaEV envelope glycoprotein” as used herein refers to the wild-type form of the BaEV envelope glycoprotein or to a mutant of said wild-type BaEV envelope glycoprotein which is at least 80%, at least 85%, at least 90%, at least 95%, or at least 99% identical to said wild-type BaEV envelope glycoprotein, provided that said mutant glycoprotein retains the capacity of the wild-type glycoprotein of binding to and fusing with hematopoietic cells membrane.

Preferably, the wild-type BaEV envelope glycoprotein consists of the sequence SEQ ID NO: 1. As known from the skilled person, the BaEV envelope glycoprotein is constituted by a cytoplasmic tail domain, a transmembrane domain and an extracellular domain. The regions corresponding to the cytoplasmic tail domain, the transmembrane domain and extracellular domain in the envelope glycoprotein sequence can be easily determined by the skilled person. Typically, the cytoplasmic tail domain is located between amino acids 530 to 564 of the wild-type BaEV envelope glycoprotein. Typically, the transmembrane domain is located between amino acids 507 to 529 of the wild-type BaEV envelope glycoprotein. Typically, the extracellular domain is located between amino acids 1 to 506 of the wild-type BaEV envelope glycoprotein.

In a particular embodiment of the invention, the cytoplasmic tail domain of the BaEV envelope glycoprotein is devoid of the fusion inhibitory R peptide.

In the context of the invention, the expression “fusion inhibitory R peptide” refers to the C-terminal portion of the cytoplasmic tail domain of the envelope glycoprotein which harbors a tyrosine endocytosis signal—YXXL—and which is cleaved by viral protease during virion maturation, thus enhancing membrane fusion of the envelope glycoprotein. The fusion inhibitory R peptide of the BaEV envelope glycoprotein is typically located between amino acids 547 and 564 of the wild-type BaEV envelope glycoprotein.

Therefore, in a particularly preferred embodiment, the modified BaEV envelope glycoprotein wherein the cytoplasmic tail domain is devoid of the fusion inhibitory R peptide comprises or consists in the amino acid sequence SEQ ID NO:2.

In another particular embodiment, the cytoplasmic tail domain of the BaEV envelope glycoprotein is replaced by the cytoplasmic tail domain of a murine leukemia virus (MLV) envelope glycoprotein.

The Murine Leukemia Virus envelope glycoprotein is preferably that of strain 4070A.

In the context of the invention, the term “MLV envelope glycoprotein” refers to the wild-type form of the MLV envelope glycoprotein or to a mutant of said wild-type MLV envelope glycoprotein which is at least 80%, at least 85%, at least 90%, at least 95%, at least 99% identical to said wild-type MLV envelope glycoprotein, provided that said mutant glycoprotein retains the capacity of the wild-type envelope glycoprotein of interacting with viral core proteins, in particular with lentiviral core proteins.

The region corresponding to the cytoplasmic tail domain in the envelope glycoprotein sequence can be easily determined by the skilled person. Typically, the cytoplasmic tail domain of the MLV envelope glycoprotein is located between amino acids 622 and 654 of the wild-type MLV envelope glycoprotein.

Therefore, in a particularly preferred embodiment, the chimeric envelope glycoprotein which comprises or consists in a fusion of the transmembrane and extracellular domain of a BaEV envelope glycoprotein and the cytoplasmic tail domain of a MLV envelope glycoprotein comprises or consists in the amino acid sequence SEQ ID NO: 3.

As intended herein “transferring” relates to the capacity of the vector particle or virus-like particle to initially deliver the biological material, the one or more nucleic acid sequences to the membrane or the cytoplasm of the target cell, upon being bound to the target cell. Herein, usually the target cell is a γδ T cell.

The terms “Titer” or “transduction efficiency” is used as a means to characterize and compare vector particles with regard to their ability to transduce their target cells. Thus, vector particles having an “increased titer” or an “increased transduction efficiency” are able to transduce a higher number of cells at a given vector particle volume than other vector particles with the same volume.

The multiplicity of infection (MOI) is the ratio of agents (e.g. phage or more generally virus, bacteria) to infection targets (e.g. cell). For example, when referring to a group of cells inoculated with virus particles, the MOI is the ratio of the number of virus particles to the number of target cells present in a defined space. For example, if one million virus particles are added to one million cells, the MOI is one.

The international unit (IU) is a unit used to measure the activity of vitamins, hormones, enzymes, and drugs. An IU is the amount of a substance that has a certain chemical or biological effect during a predefined time and under standardized conditions of temperature, pH, etc. When applied to cytokines, it is calculated using cell proliferation in the presence of this cytokine and normalized to an internal standard from the World Health Organization (WHO).

International Units (IU) are used to achieve comparability in the activity of cytokines in an internationally agreed manner, harmonizing and reducing variability in results, across multiple laboratories. IU are calculated using a validated assay in which the cytokine of interest is tested side by side with the defined World Health Organization (WHO) standard obtained from the National Institute for Biological Standards and Control (NIBSC). The activity of the cytokine is then normalized using this standard.

As used herein, the term “expansion” or “proliferation” refers to cell growth and multiplication of cell numbers.

The term “closed system” as used herein refers to any closed system which reduces the risk of cell culture contamination while performing culturing processes such as the introduction of new material, e.g. by transduction, and performing cell culturing steps such as proliferation, differentiation, activation, separation of cells, and/or electroporation if an in-line electroporation unit is connected. Such a system allows to operate under GMP or GMP-like conditions (“sterile”) resulting in cell compositions which are clinically applicable. Herein exemplarily the CliniMACS Prodigy® (Miltenyi Biotec B.V. & Co. KG, Germany) connected to the CliniMACS® Electroporator (Miltenyi Biotec B.V. & Co. KG, Germany) is used as a closed system. The CliniMACS Prodigy® is disclosed in WO2009/072003. But it is not intended to restrict the use of the method of the present invention to the CliniMACS Prodigy®. The process of the invention may be performed in a closed system, comprising a centrifugation chamber comprising a base plate and cover plate connected by a cylinder, pumps, valves, a magnetic cell separation column and a tubing set. The blood samples or other sources comprising T cells may be transferred to and from the tubing set by sterile docking or sterile welding. A suitable system is disclosed in WO2009/072003.

The closed system may comprise a plurality of tubing sets (TS) where cells are transferred between TS by sterile docking or sterile welding.

Different modules of the process may be performed in different functionally closed TS with transfer of the product (cells) of one module generated in the one tubing set to another tubing set by sterile means. For example, T cells can be magnetically enriched in a first tubing set (TS) TS100 by Miltenyi Biotec and the positive fraction containing enriched T cells is welded off the TS100 and welded onto a second tubing set TS730 by Miltenyi Biotec for further activation, modification, cultivation and washing.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® as disclosed herein.

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising γδ T cells and αβ T cells may be provided in transfer bags or other suited containers which can be connected to the closed system by sterile means.

The term “particle” as used herein refers to a solid phase such as colloidal particles, microspheres, nanoparticles, or beads. Methods for generation of such particles are well known in the field of the art. The particles may be magnetic particles. The particles may be in a solution or suspension or they may be in a lyophilised state prior to use in the present invention. The lyophilized particle is then reconstituted in convenient buffer before contacting the sample to be processed regarding the present invention.

The term “magnetic” in “magnetic particle” as used herein refers to all subtypes of magnetic particles which can be prepared with methods well known to the skilled person in the art, especially ferromagnetic particles, superparamagnetic particles and paramagnetic particles.

For enrichment, isolation or selection in principle any sorting technology can be used. This includes for example affinity chromatography or any other antibody-dependent separation technique known in the art. Any ligand-dependent separation technique known in the art may be used in conjunction with both positive and negative separation techniques that rely on the physical properties of the cells. An especially potent sorting technology is magnetic cell sorting. Methods to separate cells magnetically are commercially available e.g. from Invitrogen, Stem cell Technologies, in Cellpro, Seattle or Advanced Magnetics, Boston. For example, monoclonal antibodies can be directly coupled to magnetic polystyrene particles like Dynal M 450 or similar magnetic particles and used e.g. for cell separation. The Dynabeads technology is not column based, instead these magnetic beads with attached cells enjoy liquid phase kinetics in a sample tube, and the cells are isolated by placing the tube on a magnetic rack. However, in a preferred procedure for enriching or depleting cells from a sample comprising different types of cells antibodies or antigen binding fragments thereof may be used in conjunction with colloidal superparamagnetic microparticles having an organic coating by e.g. polysaccharides (Magnetic-activated cell sorting (MACS) technology (Miltenyi Biotec B.V. & Co. KG, Germany)). These particles (nanobeads or MicroBeads) can be either directly conjugated to monoclonal antibodies or used in combination with anti-immunoglobulin, avidin or anti-hapten-specific MicroBeads.

The MACS technology allows cells to be separated by incubating them with magnetic nanoparticles coated with antibodies directed against a particular surface antigen. This causes the cells expressing this antigen to attach to the magnetic nanoparticles. Afterwards the cell solution is transferred on a column placed in a strong magnetic field. In this step, the cells attach to the nanoparticles (expressing the antigen) and stay on the column, while other cells (not expressing the antigen) flow through. With this method, the cells can be separated positively or negatively with respect to the particular antigen(s)/marker(s).

In case of a positive selection the cells expressing the antigen(s) of interest, which attached to the magnetic column, are washed out to a separate vessel, after removing the column from the magnetic field.

In case of a negative selection the antibody used is directed against surface antigen(s) which are known to be present on cells that are not of interest. After application of the cells/magnetic nanoparticles solution onto the column the cells expressing these antigens bind to the column and the fraction that goes through is collected, as it contains the cells of interest. As these cells are non-labelled by an antibody coupled to nanoparticles, they are “untouched”.

The procedure can be performed using direct magnetic labelling or indirect magnetic labelling. For direct labelling the specific antibody is directly coupled to the magnetic particle. Indirect labelling is a convenient alternative when direct magnetic labelling is not possible or not desired. A primary antibody, a specific monoclonal or polyclonal antibody, a combination of primary antibodies, directed against any cell surface marker can be used for this labelling strategy. The primary antibody can either be unconjugated, biotinylated, or fluorophore-conjugated. The magnetic labelling is then achieved with anti-immunoglobulin MicroBeads, anti-biotin MicroBeads, or anti-fluorophore MicroBeads.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity. The tumor associated antigen (TAA) as used herein refers to an antigenic substance produced in tumor cells. Tumor associated antigens are useful tumor or cancer markers in identifying tumor/cancer cells with diagnostic tests and are potential candidates for use in cancer therapy. Preferentially, the TAA may be expressed on the cell surface of the tumor/cancer cell, so that it may be recognized by the antigen binding receptor as disclosed herein.

The term “antigen-binding molecule” as used herein refers to any molecule that binds preferably to or is specific for the desired target molecule of the cell, i.e. the antigen. The term “antigen-binding molecule” comprises e.g. an antibody or antigen binding fragment thereof. The term “antibody” as used herein refers to polyclonal or monoclonal antibodies, which can be generated by methods well known to the person skilled in the art. The antibody may be of any species, e.g. murine, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “antibody” comprises both intact molecules and antigen binding fragments, such as Fab, Fab′, F(ab′)2, Fv, nanobodies and single-chain antibodies. Additionally, the term “antigen-binding fragment” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g. receptor-ligand interaction). The linkage (coupling) between antibody and particle or nanostructure can be covalent or non-covalent. A covalent linkage can be, e.g. the linkage to carboxyl-groups on polystyrene beads, or to NH2 or SH2 groups on modified beads. A non-covalent linkage is e.g. via biotin-avidin or a fluorophore-coupled-particle linked to anti-fluorophore antibody.

The terms “specifically binds to” or “specific for” with respect to an antigen-binding molecule, e.g. an antibody or antigen-binding fragment thereof, refer to an antigen-binding molecule (in case of an antibody or antigen-binding fragment thereof to an antigen-binding domain) which recognizes and binds to a specific antigen in a sample, e.g. CD4, but does not substantially recognize or bind other antigens in said sample. An antigen-binding domain of an antibody or antigen-binding fragment thereof that binds specifically to an antigen from one species may bind also to that antigen from another species. This cross-species reactivity is not contrary to the definition of “specific for” as used herein. An antigen-binding domain of an antibody or antigen-binding fragment thereof that specifically binds to an antigen, e.g. the CD4 antigen, may also bind substantially to different variants of said antigen (allelic variants, splice variants, isoforms etc.). This cross reactivity is not contrary to the definition of that antigen-binding domain as specific for the antigen, e.g. for CD4.

The terms “genetically modified immune cell” or “engineered immune cell” may be used interchangeably and mean containing and/or expressing a foreign gene or nucleic acid sequence which in turn modifies the genotype or phenotype of the cell or its progeny. Especially, the terms refer to the fact that cells can be manipulated by recombinant methods well known in the art to express stably or transiently peptides or proteins, e.g. CARs which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

As used herein “autologous” means that cells, a cell line, or population of cells used for treating subjects are originating from said subject.

As used herein “allogeneic” means that cells or population of cells used for treating subjects are not originating from said subject but from a donor.

The terms “immune cell” or “immune effector cell” may be used interchangeably and refer to a cell that may be part of the immune system and executes a particular effector function such as T cells, alpha-beta T cells, gamma-delta T cells, NK cells, NKT cells, B cells, innate lymphoid cells (ILC), cytokine induced killer (CIK) cells, lymphokine activated killer (LAK) cells, gamma-delta T cells, regulatory T cells (Treg), monocytes or macrophages. Preferentially these immune cells are human immune cells.

Characterized by expression of a T cell receptor (TCR) composed of gamma and delta chains (γδTCR), γδ T cells are an innate-like subset of human T cells representing up to 10% of peripheral CD3-positive cells and up to 60% of intraepithelial lymphocytes in healthy donors. Two subsets composed of Vγ9Vδ2-TCR and VγxVδI-TCR-expressing cells (where x denotes one of 6 functional gamma chain genes) dominate the peripheral γδ T cell compartment. In contrast to the oligoclonal and phosphoantigen reactive Vγ9Vδ2-TCR population, VγxVδ1 cells (referred to also as ‘Vδ1 cells’) express a Vδ1-TCR chain paired with one of various Vγ-chains. Normally, the term “γδ T cells” as used herein refer to both Vδ1 cells and Vγ9Vδ2 cell. Mainly the Vγ9Vδ2 cells expand using the expansion as disclosed herein. Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR cell immunotherapy” or in case of use of T cells only as “T cell therapy” or “CAR T cell immunotherapy”, when these cells express a CAR.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription and/or translation of a particular nucleotide sequence driven by its promoter in a cell.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The terms “nucleic acid”, “nucleic acid sequence/molecule’” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “basal medium for animal or human cells” as used herein refers to a defined synthetic medium for animal or human cells that is buffered preferably at a pH between 7.2 and 7.6, preferentially at about a pH of 7.4 with a carbonate-based buffer, while the cells are cultured in an atmosphere comprising between 5% and 10% CO2, preferably about 5% CO2.

In blood, the serum is the component that is neither a blood cell (serum does not contain white blood cells-leukocytes, or red blood cells—erythrocytes), nor a clotting factor; it is the blood plasma not including the fibrinogens. Serum includes all proteins not used in blood clotting and all the electrolytes, antibodies, antigens, hormones, and any exogenous substances. Human serum is the serum from a human.

The term “feeder cells” refers to cells that are added to a culture of target cells to support their survival and/or growth. Feeder cells provide an intact and functional extracellular matrix and matrix-associated factors and secrete known and unknown cytokines into the conditioned medium. Feeder cells are usually growth arrested to prevent their proliferation in the culture, but their survival may be maintained. Growth arrest can be achieved by irradiation with an effective dose or treatment with an effective dose of chemicals such as Mytomycin C.

The term “activation” as used herein refers to inducing physiological changes with a cell that increase target cell function, proliferation and/or differentiation.

The term “transduction” means the transfer of genetic material from a viral agent such as a lentiviral vector particle into a eukaryotic cell such as a T-cell.

The term “isolated” is used herein to indicate that the polypeptide, nucleic acid or host cell exist in a physical milieu distinct from that in which it occurs in nature. For example, the isolated polypeptide may be substantially isolated (for example enriched or purified) with respect to the complex cellular milieu in which it naturally occurs, such as in a crude extract.

A transgene may be a gene that has been transferred by genetic engineering techniques into a host that normally does nor bear this gene. The gene may be a naturally gene that occurs in other cells or may be a recombinant gene. Most prominent transgenes used in the present invention may be the T cell receptor and the chimeric antigen receptor.

The T cell receptor (TCR) is a protein complex found on the surface of T cells, or T lymphocytes, that is responsible for recognizing fragments of antigen as peptides bound to major histocompatibility complex (MHC) molecules.

In general, a CAR as used herein may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell. Such a CAR may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target. The tag may be e.g. dextran or a hapten such as biotin or fluorescein isothiocyanate (FITC) or phycoerythrin (PE) or thiamin, but the tag may also be a peptide sequence e.g. chemically or recombinantly coupled to the polypeptide part of the tagged polypeptide. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G4/S)3-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge. The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

If the CAR is an inhibitory CAR (referred to herein normally as “iCAR”) that may be expressed in addition to an activating CAR as described above in a cell, then said iCAR may have the same extracellular and/or transmembrane domains as the activating CAR but differs from the activating CAR with regard to the endodomain.

The at least one endodomain of the inhibitory CAR may be a cytoplasmic signaling domain comprising at least one signal transduction element that inhibits an immune cell or comprising at least one element that induces apoptosis.

Inhibitory endodomains of an iCAR are well-known in the art and have been described e.g. in WO2015075469A1, WO2015075470A1, WO2015142314A1, WO2016055551A1, WO2016097231A1, WO2016193696A1, WO2017058753A1, WO2017068361A1, WO2018061012A1, and WO2019162695A1.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein.

The term “tagged polypeptide” as used herein refers to a polypeptide that has bound thereto directly or indirectly at least one additional component, i.e. the tag. The tagged polypeptide as used herein is able to bind an antigen expressed on a target cell. The polypeptide may be an antibody or antigen binding fragment thereof that binds to an antigen expressed on the surface of a target cell such as a tumor associated antigen on a cancer cell. The polypeptide of the tagged polypeptide alternatively may a cytokine or a growth factor or another soluble polypeptide that is capable of binding to an antigen of a target cell.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv. A kit may comprise a container with components within the container. Such containers may be e.g. boxes, bottles, vials, tubes, bags, pouches, blister packs, or other suitable container forms known in the art. Such containers may be made of plastic, glass, laminated paper, metal foil, or other materials suitable for holding components therein. The kit further may comprise written directions for using the components of the kits.

### EXAMPLES

The following examples are intended for a more detailed explanation of the invention but without restricting the invention to these examples.

### Example 1: Optimization of the Transduction Efficiency on γδ T Cells with Lentivirus Vectors in Design of Experiment (DoE)

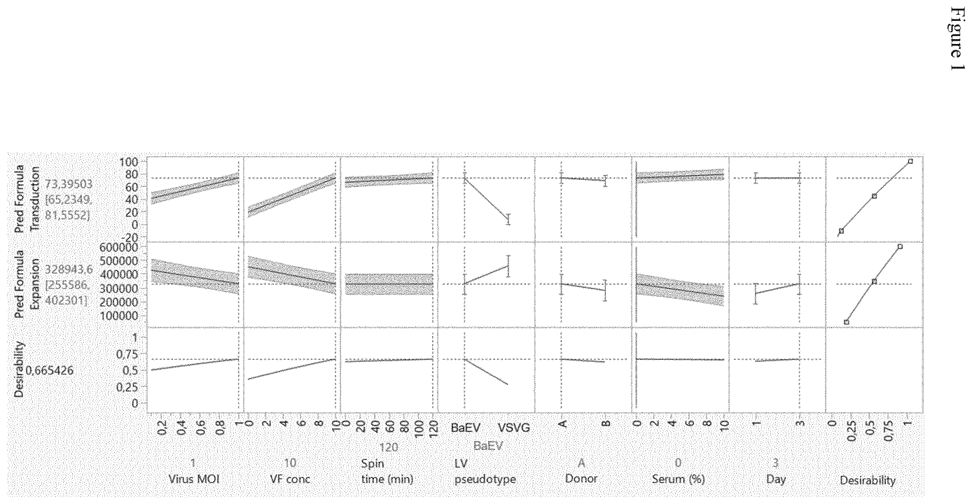

A custom DoE design was prepared with the software JMP 15.2 (SAS GmbH, Germany). Runs were randomized. Peripheral blood mononuclear cells (PBMC) from healthy donors were isolated with Pancoll (Pan biotech) density gradient centrifugation, and activated with Zometa 5 μM (Novartis), IL-2 100 IU/ml and IL-15 100 IU/ml (Miltenyi Biotec) in TexMACS™ medium (Miltenyi Biotec) with different concentration of human AB serum (0/5/10%, Access Cell Culture). At day 1 and day 3, we plated the PBMC at 2×10⁵ cells per well in a 96 well plate in serum free medium, supplemented with IL-2 and IL-15. They were then transduced with BaEV- or VSV-G-LV encoding CD19 CAR at a MOI of 0.1 to 1 in the presence of 0-10 μg/ml Vectofusin-1 (Miltenyi Biotec). Samples were then either directly incubated at 37° C., 5% CO₂ or either first centrifuged for 60/120 minutes at 400 g, 32° C. (“spinoculation”). The next day, one third in volume of medium with 0/5/10% human AB serum was added. Two days after, ⅔ of the medium was exchanged. CAR expression and γδ T cell number were determined 7 days after transduction by using biotinylated CD19 CAR Detection Reagent, followed by anti-biotin PE, CD3-VioBlue, and anti-γδTCR APC antibodies, and 7AAD for dead cells exclusion (Miltenyi Biotec). The resulting data was analyzed with JMP. The optimal desirability in the absence of serum was obtained with the prediction profiler (FIG. 1). The donor only interacted with the time of spinoculation, while other factors were independent of the donor. The optimal transduction protocol was found to be at day 3, with the BaEV pseudotype and the highest concentration of Vectofusin-1.

### Example 2: BaEV is a Potent Pseudotype for High Transduction Efficiency of γδ T Cells at a Low MOI

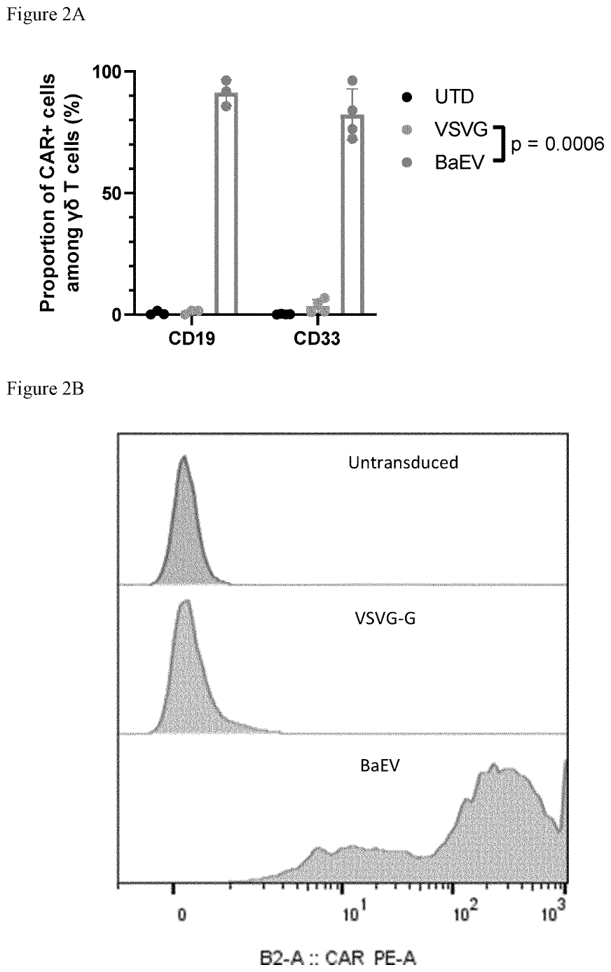

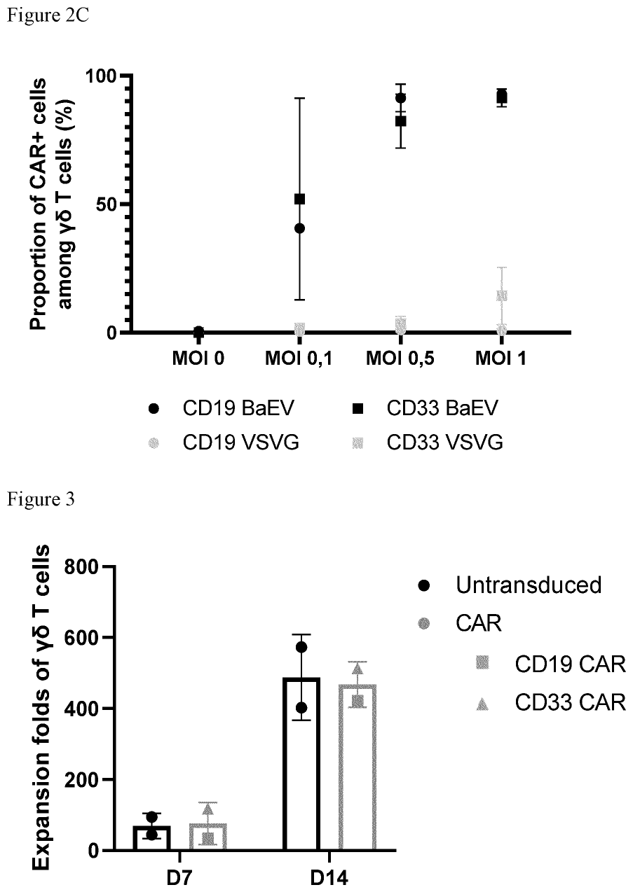

Next, we confirmed the results from the DoE by comparing CAR expression with BaEV and VSV-G pseudotyped lentiviral particles at different MOI. γδ T cells were activated and expanded for 3 days before transduction at a MOI of 0.1, 0.5 and 1. Vectofusin-1 was added to the samples transduced with BaEV-LV. CAR expression and γδ T cells number were determined 7 days after transduction by using biotinylated CD19 CAR or CD33 CAR Detection Reagent, followed by anti-biotin PE, CD3-VioBlue, anti-γδTCR APC, and anti-αβTCR APC-Vio770 antibodies, and 7AAD for dead cells exclusion (Miltenyi Biotec). The proportion of TS T cells expressing the CAR (FIG. 2A) and the Median Fluorescence Intensity (MFI) of this population (FIG. 2B) were determined at a MOI of 0.5. The transduction efficiency was also evaluated at different MOI, from 0.1 to 1 (FIG. 2C). The use of the BaEV pseudotype resulted in a significant increase in transduction efficiency at the same MOI (p=0.0006, unpaired Mann-Whitney test), reaching a plateau of at least 80% transduction efficiency at a MOI above 0.5.

### Example 3: Transducing γδ T Cells with a CAR does not Negatively Impact their Expansion and Phenotype

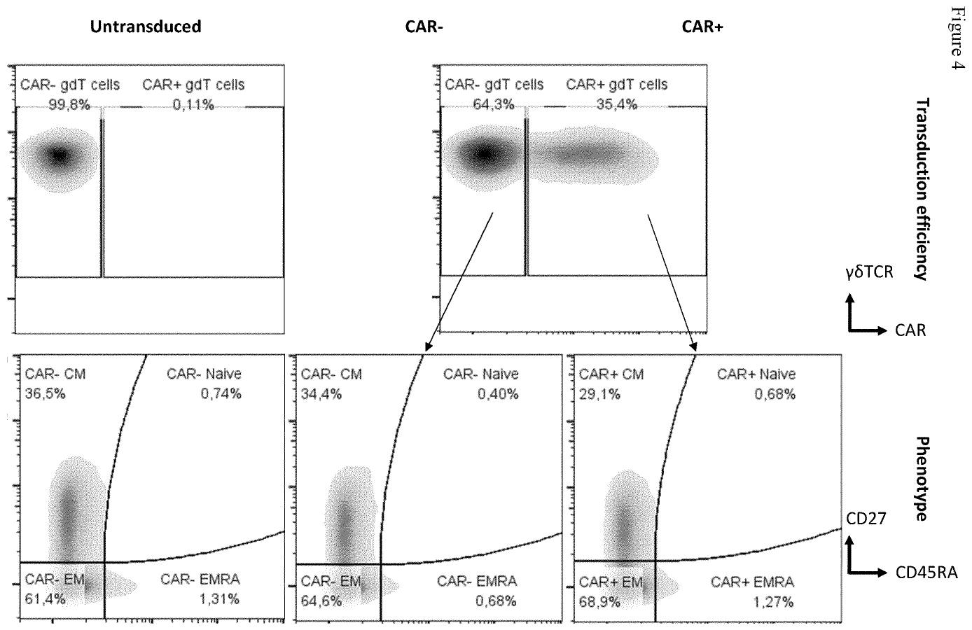

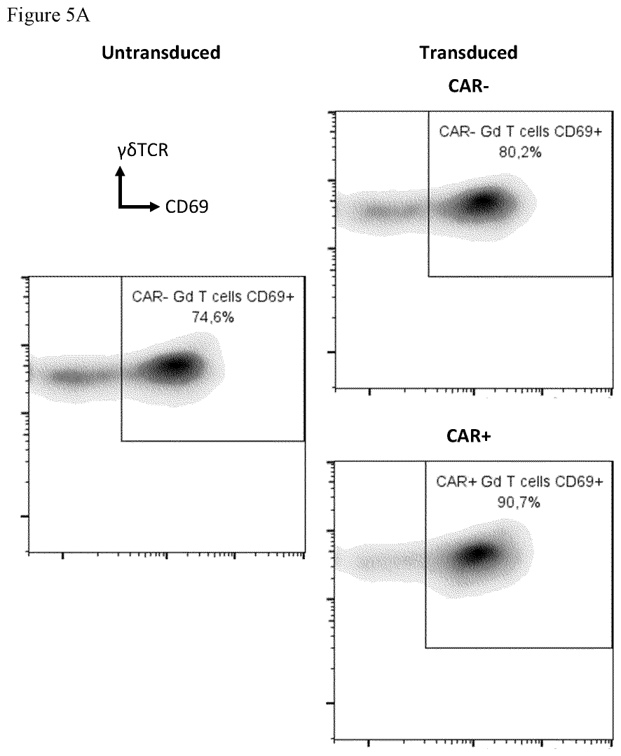

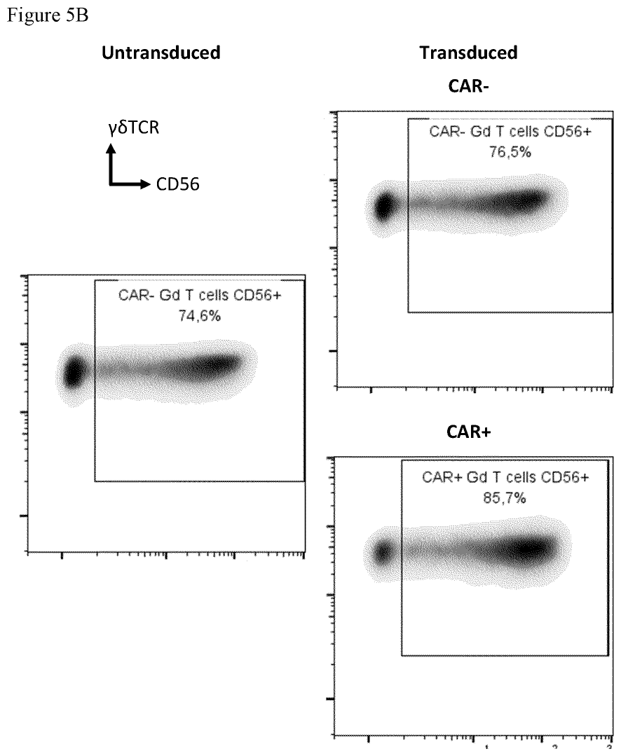

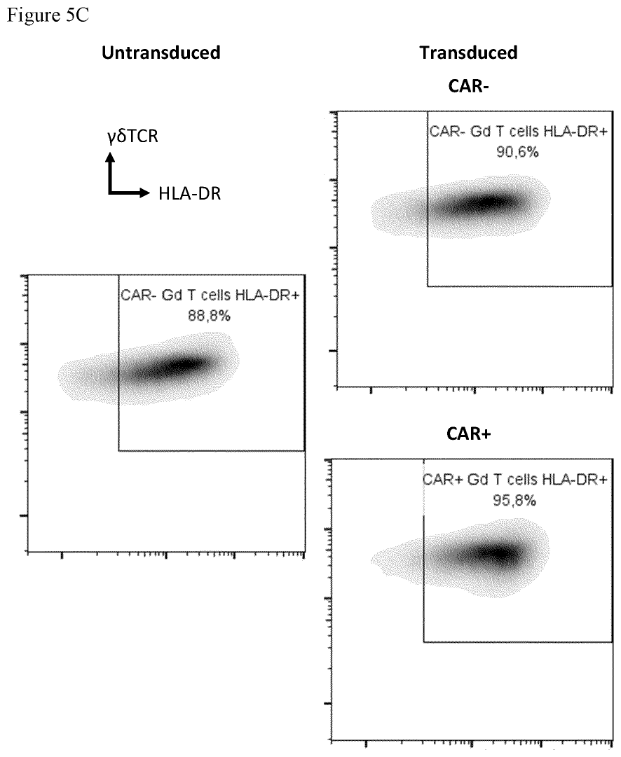

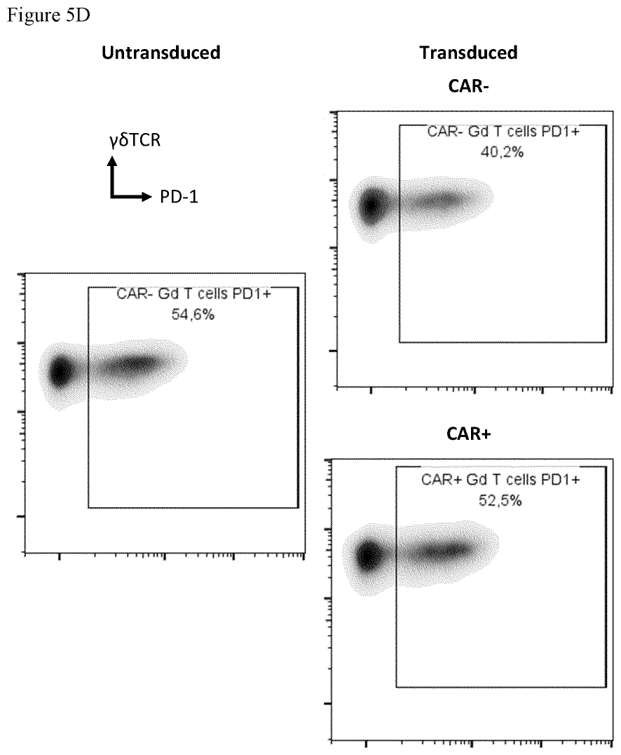

We further evaluated the expansion capacity of γδ T cells from transduced and untransduced samples in the presence of 10 μg/ml Vectofusin-1. Cells were split to stay at a density of 1×10⁶ cells/ml. The cells were counted on the MACSQuant 10, with propidium iodure (PI, Miltenyi Biotec) used for dead cells exclusion. Transduction with CD19 CAR or CD33 CAR at a MOI of 0.1 did not impact γδ T cells expansion, as measured by the expansion fold (FIG. 3). γδ T cells phenotype and activation profile at day 14 (i.e. 11 days after transduction) were then analyzed, and we compared untransduced samples to samples transduced with CD19 CAR. Two staining panels were used. The stainings were performed in the presence of FCR-blocking reagent (Miltenyi Biotec) and 7AAD was used for dead cell exclusion. Both panels included biotinylated-CD19 CAR Detection Reagent followed by anti-Biotin VioBlue and anti-γδTCR APC antibodies. The first panel further included CD56-sVioBright 515, anti-PD-1 PE, CD14-PE Vio770 and anti-αβTCR APC-Vio770 antibodies. The second panel included instead anti-HLA-DR VioGreen, CD45RA−FITC, CD27-PE and CD69-APC Vio770 antibodies (Miltenyi Biotec). At least 30% of γδ T cells were expressing CD19 CAR, which did not significantly alter their phenotype. In all samples, the main γδ T cell phenotype was effector memory (CD45RA⁻CD27⁻), followed by central memory (CD45RA⁻CD27+, FIG. 4). CD19 CAR+ γδ T cells were more activated than the CD19 CAR− cells or than untransduced samples: these samples showed the highest expression for CD69 (FIG. 5A), CD56 (FIG. 5B) and HLA-DR (FIG. 5C). However, they did not express more PD-1 than untransduced γδ T cells (FIG. 5D).

### Example 4: Transducing γδ T Cells with a CAR Results in Functional CAR γδ T Cells

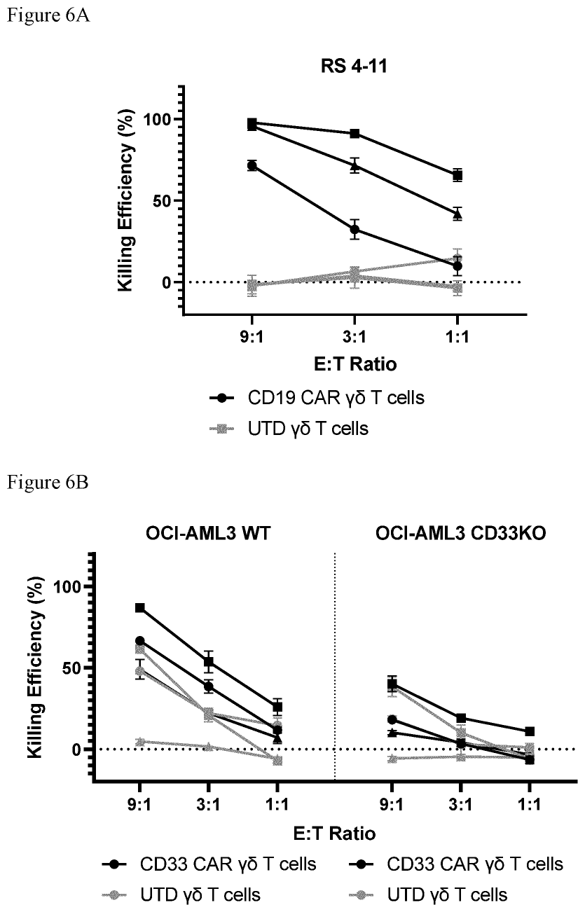

PBMCs from three donors were similarly expanded and transduced with CD19 CAR or CD33 CAR at a MOI of 0.5. The cells were depleted from αβ T cells before their activation, expansion and transduction. The cells were incubated with biotinylated anti-αβTCR antibody (Miltenyi Biotec) for 30 minutes, washed twice and then incubated with anti-biotin microbeads (Miltenyi Biotec) for 30 minutes. The cells were passed through a column in a magnetic field, in which αβ T cells were retained and thus depleted from the sample. Cells were then plated in TexMACS™ medium (Miltenyi Biotec) supplemented with IL-2 and IL-15 (Miltenyi Biotec), and activated with Zoledronate 5 μM (Novartis Pharma AG). They were transduced three days after activation by BaEV-LV containing either a CD19-CAR sequence or a CD33-CAR sequence. After ten more days of expansion, untransduced and CD19 CAR transduced cells were cocultured for four hours at different effector to target (E:T) ratios with GFP+ Luciferase+ RS4,11 cancer cells (FIG. 6A) while untransduced and CD33 CAR transduced cells were cocultured with GFP+ Luciferase+ OCI-AML3 WT and GFP+ Luciferase+ OCI-AML3 CD33 KO cancer cells (FIG. 6B). Half of the supernatant was discarded and replaced by OneGlo reagent (Promega), which contains luciferin, a lysis buffer and all the components required for luciferase activity. The luminescence was measured on the plate reader Victor3 (PerkinElmer) and the percentage of killing was obtained by dividing the luminescence of cocultured cells with the luminescence from target cells alone. The transduction with the CAR increased the killing efficiency of the γδ T cells in presence of its antigen target.

### Example 5: γδ T Cells are Efficiently Expanded with Zoledronate, IL-2 and IL-15 in the Presence of Human Serum

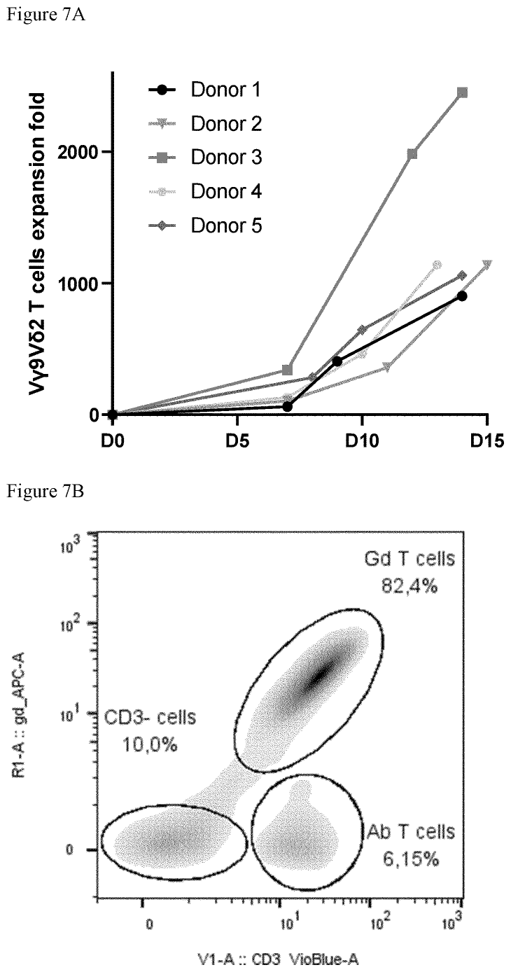

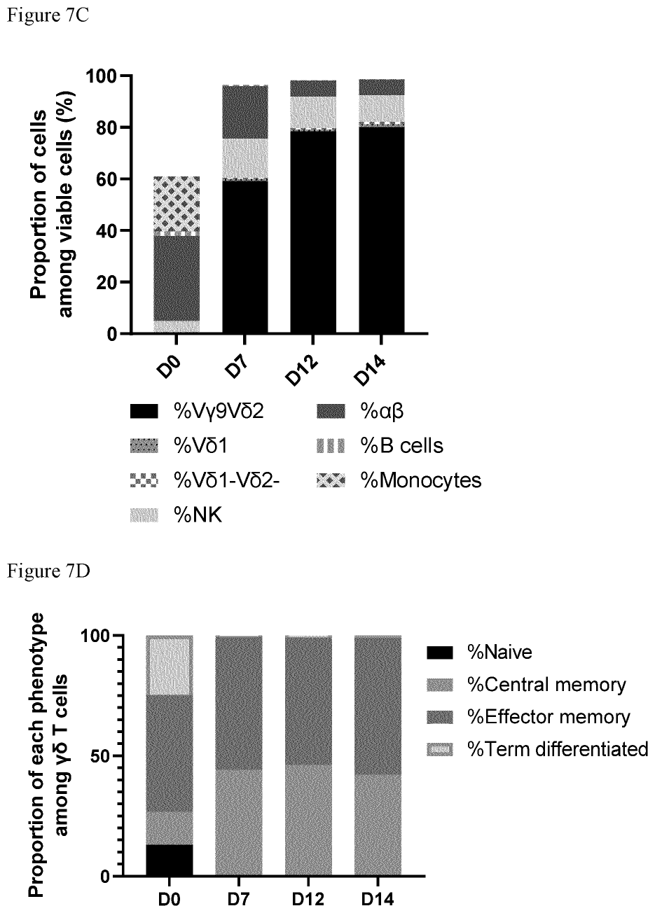

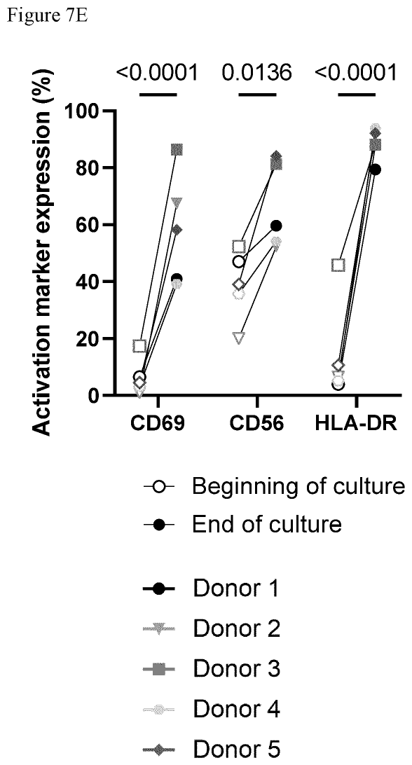

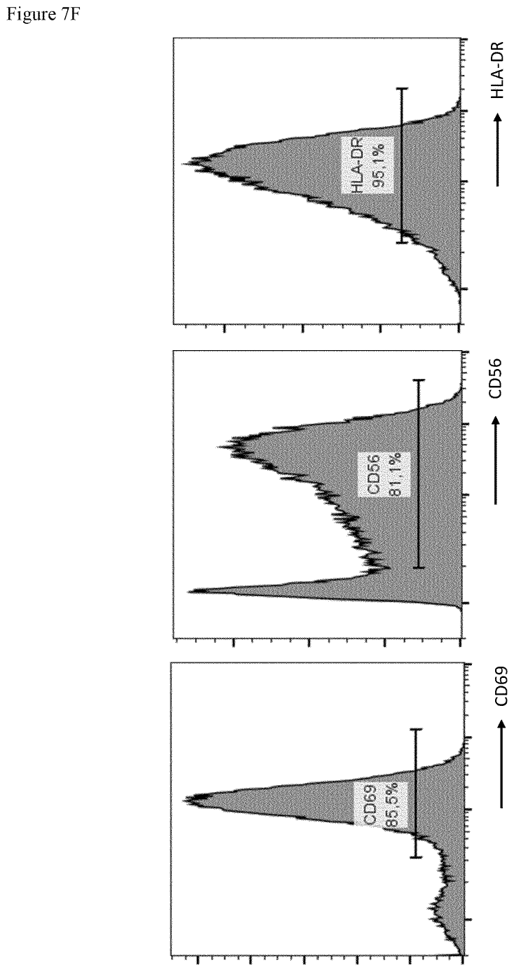

PBMC were activated with Zometa 5 μM (Novartis Pharma AG), IL-2 100 IU/ml and IL-15 100 IU/ml in TexMACS™ medium (Miltenyi Biotec) in the presence of 10% human AB serum (Access Cell Culture). The cellular composition, phenotype and activation profile were verified throughout the culture with flow cytometry until two weeks of culture. The following antibodies coupled to fluorophores from Miltenyi Biotec were used: CD3, CD56, CD14, CD19, anti-γδTCR, anti-Vd1, anti-Vd2, anti-Vg9, CD27, CD45RA, CD69 and anti-HLA-DR. The staining was performed in presence of FCR blocking reagent, and 7AAD was used for dead cell exclusion. A Vγ9Vδ2 T cell expansion of at least 1000 folds was observed in different donors (FIG. 7A), with a γδ T cells purity above 80% (representative density plot shown in FIG. 7B). Throughout the culture, the proportion of monocytes (CD14+), B cells (CD19+) decreased while the proportion of NK cells (CD3− CD56+) and especially Vγ9Vδ2 T cells increased (representative graph in FIG. 7C). The phenotype of γδ T cells evolved from a heterogeneous phenotype to a well-defined effector memory phenotype (representative graph in FIG. 7D). All activation markers measured (CD69, CD56 and HLA-DR) were upregulated at the end of the culture (FIG. 7E, statistical analysis performed with Šidák's multiple comparisons test, representative density plots in FIG. 7F). After two weeks of culture, the γδ T cells had greatly expanded and had a favorable phenotype and extracellular markers expression for cellular immunotherapies.

### Example 6: Cell Culture in Serum-Free Conditions Preserve a High Expansion Fold and a Favorable Phenotype for their Use in Therapy

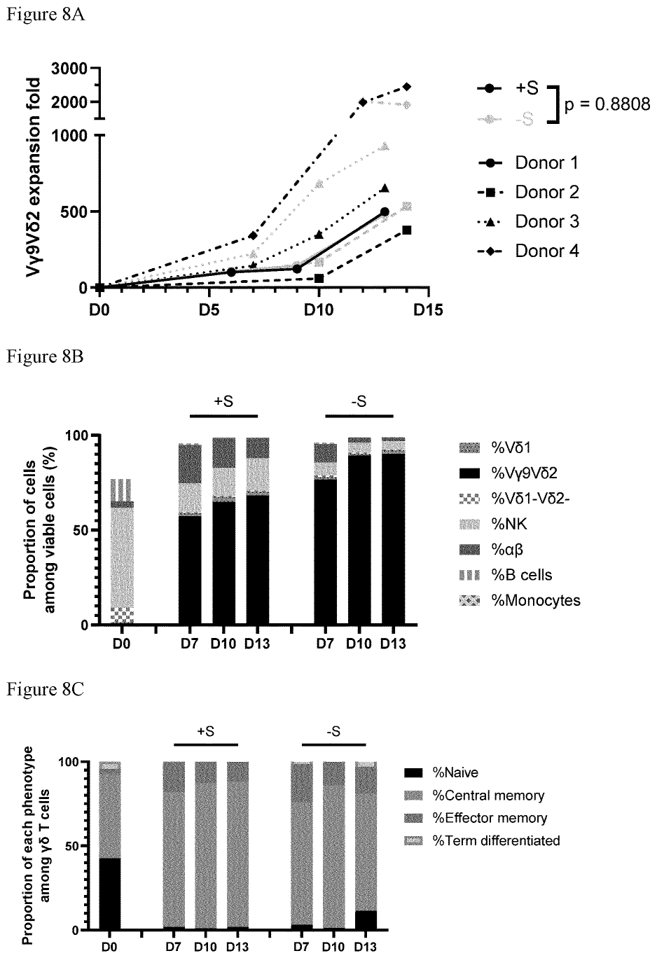

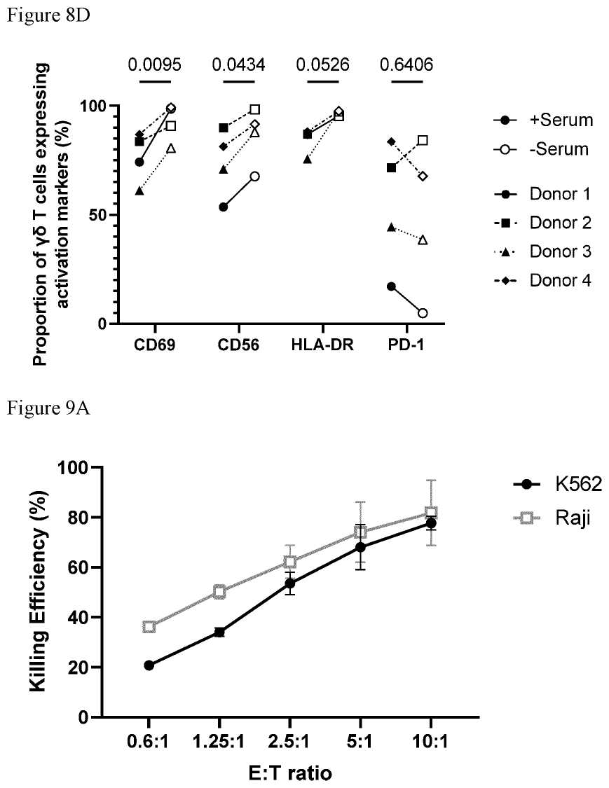

Peripheral blood mononuclear cells (PBMC) were activated with Zometa 5 μM (Novartis Pharma AG), IL-2 100 IU/ml and IL-15 100 IU/ml in TexMACS™ medium (Miltenyi Biotec), this time in the absence or presence of 10% human AB serum (Access Cell Culture). The cellular composition, phenotype and activation profile were verified throughout the culture with flow cytometry until two weeks of culture. The following antibodies coupled to fluorophores from Miltenyi Biotec were used: CD3, CD56, CD14, CD19, anti-γδTCR, anti-Vd1, anti-Vd2, anti-Vg9, CD27, CD45RA, CD69 and anti-HLA-DR. The staining was performed in presence of FCR blocking reagent, and 7AAD was used for dead cells exclusion. The Vγ9Vδ2 T cells expanded similarly in the presence and absence of human serum in 4 donors (FIG. 8A, p=0.8808, paired t-test), with an increase in γδ T cells purity in the absence of human serum (representative graph shown in FIG. 8B). In particular, the proportion of NK cells and αβ T cells decreased in the absence of serum. The phenotype of γδ T cells was similar in both conditions, with a majority of central and effector memory cells after one week of culture (representative graph in FIG. 8C). The activation markers measured (CD69, CD56 and HLA-DR) were upregulated in the absence of serum (FIG. 8D, Šidák's multiple comparisons tests gave p=0.095 for CD69, p=0.0434 for CD56), though it was not significant for HLA-DR (p=0.0526) at the end of the culture. PD-1 was not significantly affected by the absence of serum, though it seemed down regulated in 3 out of 4 donors. Thus, the absence improved the cell product characteristics by increasing the purity in γδ T cells and increasing their activation without impacting their expansion or phenotype.

### Example 7: γδ T Cells Expanded with Our Method Demonstrated Functionality In-Vitro by Different Cytotoxicity Assays

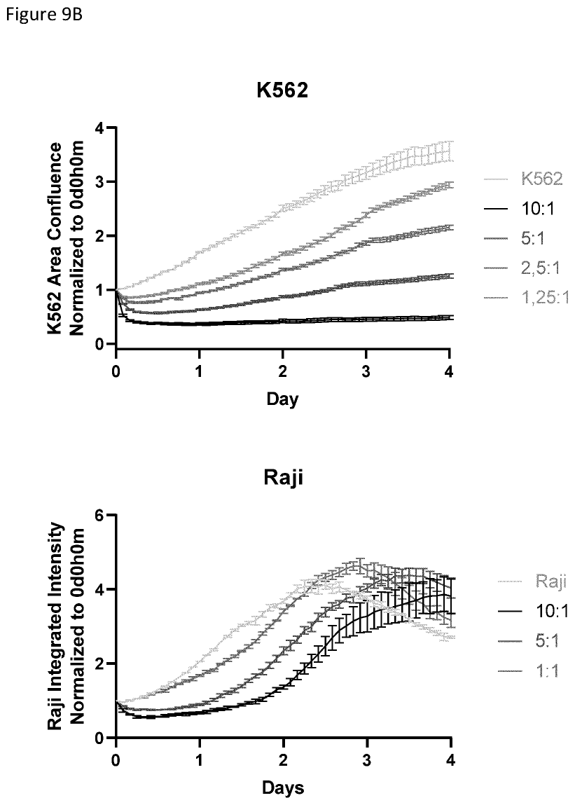

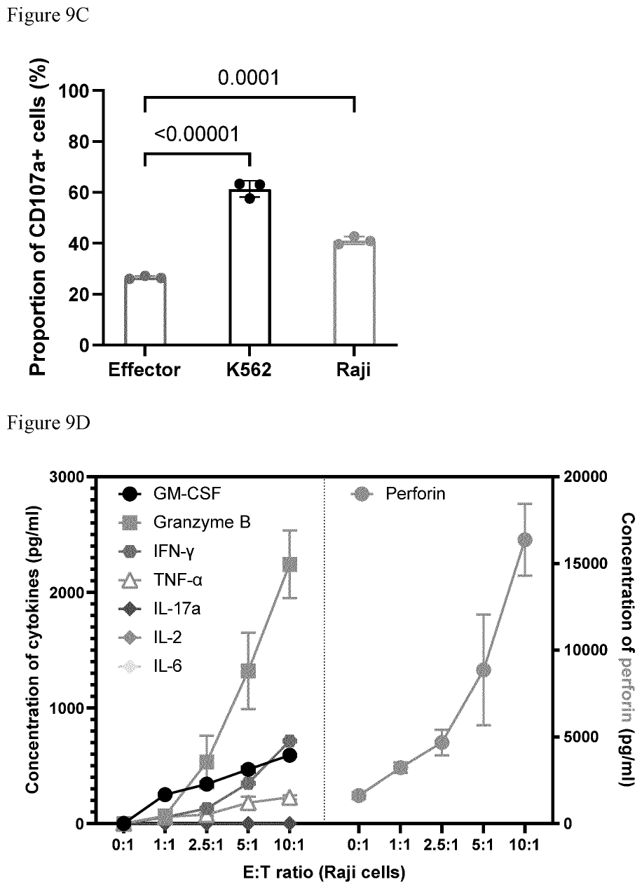

γδ T cells were expanded from PBMC for 10 days, and then isolated with the anti-TCRγ/δ MicroBead Kit (Miltenyi Biotec). They were further cultured for 2 to 4 days. Their cytotoxicity against the cancer cells K562 and Raji were tested in several assays. Their direct cytotoxicity against these cancer cell lines was first verified in the flow-cytometry assay and fluorescence-based Incucyte assay. The γδ T cells, also termed effector cells, were cocultured with GFP+ K562 or Raji cells, also termed target cells, at different effector to target ratios (E:T ratios) in the cancer cell growth medium. The number of living cells was determined after 4 h of coculture by flow cytometry, using PI for live/dead cell gating and gating on GFP+ cells for the target cells. It was then compared to target cells alone to determine the killing efficiency (FIG. 9A). In the Incucyte assay, the coculture was performed in the Incucyte® S3 Live-Cell Analysis System (Sartorius), with pictures taken every 2 hours for 4 days. The growth kinetics of the cancer cells was then plotted with and without effector cells (FIG. 9B). Both assays demonstrated the killing potency of untransduced γδ T cells against these two target cell lines. We then explored degranulation and cytokine secretion. We cocultured isolated untransduced γδ T cells with K562 or Raji cells for 5 h in presence of CD107a antibody (Miltenyi Biotec) including Golgi stop for 4 hours (BD Biosciences), before staining the cells and measuring the proportion of CD107a+γδ T cells (FIG. 9C). γδ T cells degranulated more in presence of cancer cells. We further evaluated secretion profile for different cytokines after 24 hours of coculture with the MACSPlex T/NK kit (FIG. 9D). The secretion of two cytotoxic molecules, perforin and granzyme B, was increased in the presence of the target cells. Other cytokines and chemokines which are antitumoral were increased: IFN-γ, TNF-α but also GM-CSF. No secretion of IL-2, IL-6 or IL-17a was observed. Taken together, these results demonstrate the good antitumoral activity of the γδ T cells expanded with our protocol.

### Example 8: The Expansion and Transduction Methods Described Herein are Compatible with the Clinical Use of Genetically Modified γδ T Cells

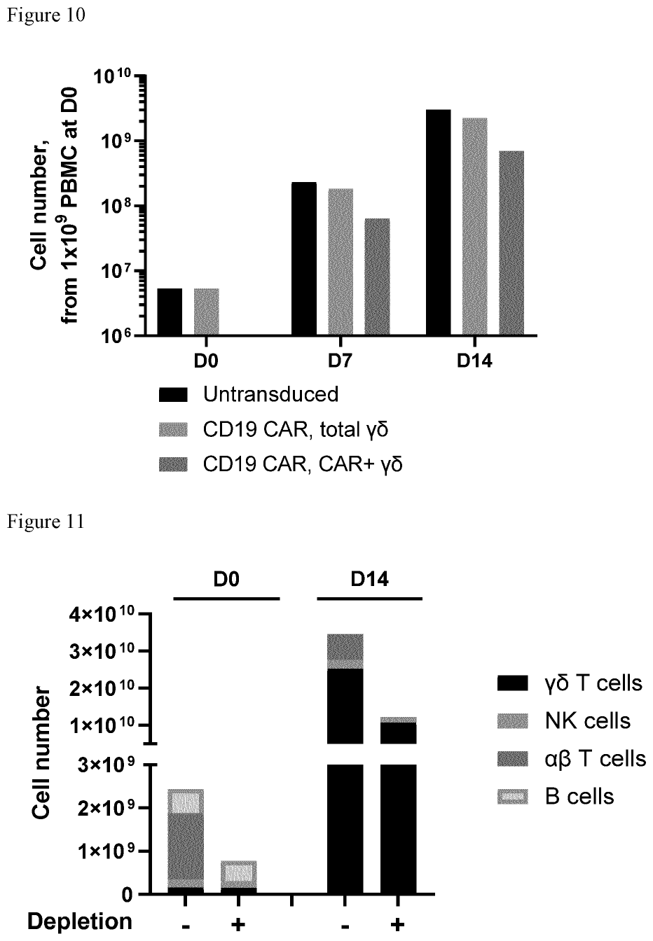

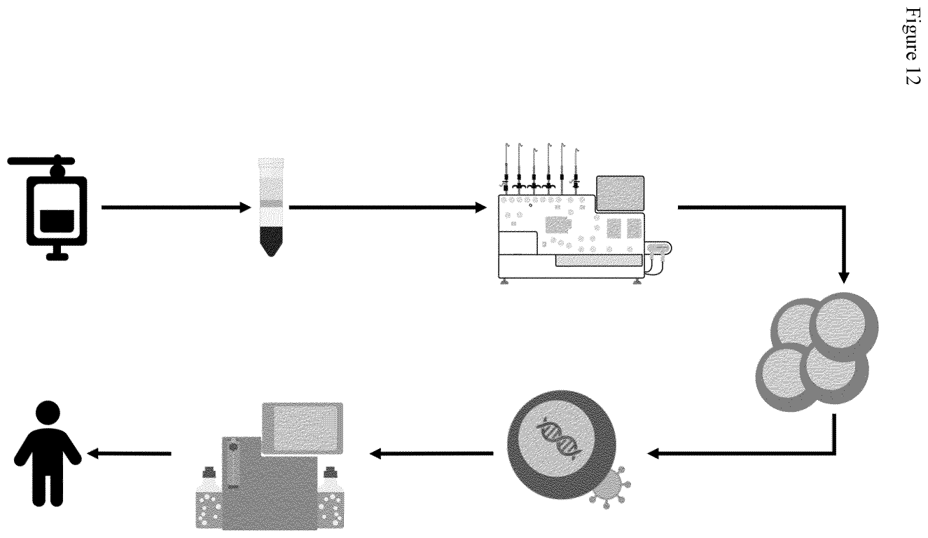

From the expansion-fold and the proportion of CD19 CAR+ γδ T cells after the activation, transduction with a BaEV-LV at a MOI of 0.1 and expansion of 1×10⁹ PBMC, the total γδ T cells number and the genetically-engineered γδ T cell number were estimated. They were compared with untransduced cells (FIG. 10). From less than 1×10⁷ γδ T cells in the initial sample, at least 1×10⁹ γδ T cells including at least 5×10⁸ CAR+ γδ T cells were obtained in two weeks. γδ T cell expansion was also evaluated when combined with the depletion of αβ T cells at the beginning of the process. After PBMC isolation, half of the cells were incubated with biotinylated anti-αβTCR antibody (Miltenyi Biotec) for 30 minutes, washed twice and then incubated with anti-biotin microbeads (Miltenyi Biotec) for 30 minutes. The cells were passed through a column in a magnetic field, in which αβ T cells were retained and thus depleted from the sample. Cells were then plated in TexMACS supplemented with IL-2, IL-15 and 10% AB serum, and activated with Zoledronate 5 μM. After two weeks of expansion, the cells were stained with 7AAD, CD3, anti-γδTCR, CD56 and CD19 and measured with flow cytometry. The proportion of each population from samples with or without αβ T cells depletion was compared (FIG. 11). The depletion allowed to remove αβ T cells by at least a log 5 at day 0, and their number stayed low throughout the expansion. We developed a process starting with PBMC isolation from peripheral blood, combined or not with αβTCR depletion. Activation with Zoledronate and expansion in low doses of IL-2 and IL-15-containing medium is followed by BaEV-pseudotypes LV transduction and further expansion of engineered cells. This process is suitable for engineered Vγ9Vδ2 T cells production in a closed and scalable platform for allogeneic clinical interventions (FIG. 12).

### Example 9: CAR γδ T Cells Expanded in the Absence of Serum Show an Increased Cytotoxic Potency

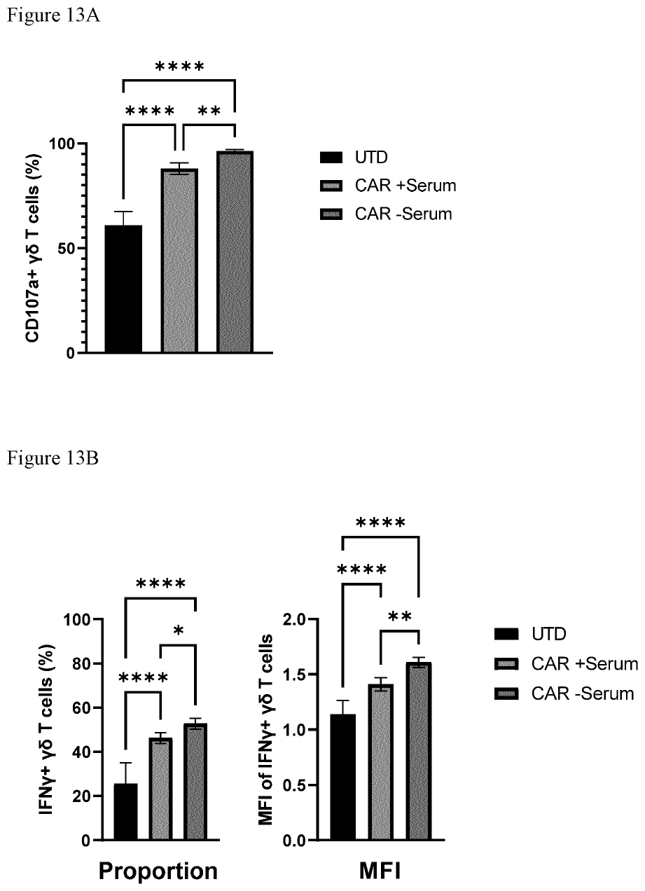

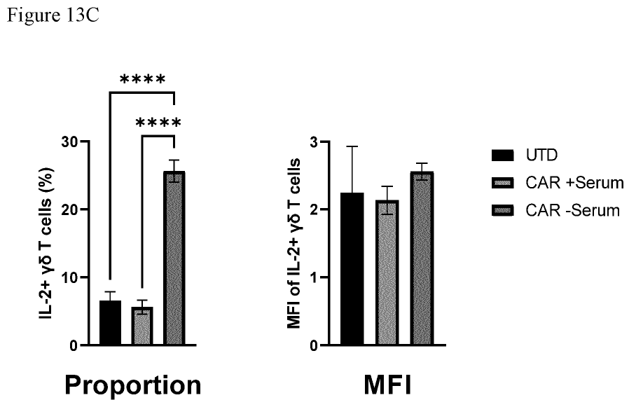

γδ T cells were expanded from PBMC after αβ T cell depletion in either serum-free medium or medium containing 5% of human AB serum. They were transduced 3 days after the beginning of the culture with CD19 CAR using BaEV lentiviral vector and Vectofusin-1 (Miltenyi Biotec). Their functionality was assessed at day 14 with degranulation assay and intracellular cytokine staining. To measure their degranulation, the cells were incubated for 2 hours in presence of CD107a-PE antibody or the corresponding isotype (Miltenyi Biotec) and bafilomycin (Sigma). They were then stained with CD3-VioBlue, γδTCR-APC and 7AAD (Miltenyi Biotec) and measured on a MACSQuant 10 (Miltenyi Biotec). For intracellular cytokine staining, the cells were first incubated 2 hours with brefeldin A (Sigma). They were stained with Viobility 405/520 (Miltenyi Biotec) and fixed and permeabilized. They were further stained with a cocktail of antibodies comprising CD3-VioBlue, γδTCR-APC, IL-2-PE Vio605 and IFNγ-APC Vio770 (Miltenyi Biotec). All γδ T cells transduced with the CD19 CAR had a higher CD107a (FIG. 13A) and IFNγ expression (FIG. 13B) than their non-transduced counterparts, whereas only CAR γδ T cells expanded in serum-free medium expressed more IL-2 than untransduced cells (FIG. 13C). Overall, CAR γδ T cells expanded in the absence of serum expressed more CD107a, IFNγ and IL-2 than CAR γδ T cells expanded in 5% human serum. Thus, the absence of serum in the medium improved the cell product characteristics by increasing their ability to degranulate and produce cytokines.

### REFERENCES

1—Groh V, Rhinehart R, Secrist H, Bauer S, Grabstein K H, Spies T. Broad tumor-associated expression and recognition by tumor-derived γδ T cells of MICA and MICB. Proc Natl Acad Sci USA (1999) 96:6879-84. doi:10.1073/pnas.96.12.6879
2—Todaro M, D'Asaro M, Caccamo N, Iovino F, Francipane M G, Meraviglia S, et al. Efficient killing of human colon cancer stem cells by γδ T lymphocytes. J Immunol (2009) 182:7287-96. doi:10.4049/jimmunol.0804288
3—Maeurer M J, Martin D, Walter W, Liu K, Zitvogel L, Halusczcak K, et al. Human intestinal Vδ+ lymphocytes recognize tumor cells of epithelial origin. J Exp Med (1996) 183(4):1681-96. doi:10.1084/jem.183.4.1681
4—Chien Y H, Bonneville M. γδ T cell receptors. Cell Mol Life Sci (2006) 63(18):2089-94. doi:10.1007/s00018-006-6020-z
5—Viney J, MacDonald T T, Spencer J. γδ T cells in the gut epithelium. Gut (1990) 31(8):841-4. doi:10.1136/gut.31.8.841
6—Hohlfeld R, Engel A G, Ii K, Harper M C. Polymyositis mediated by T lymphocytes that express the γδ receptor. N Engl J Med (1991) 324(13):877-81. doi:10.1056/NEJM199103283241303
7—Hayday A, Tigelaar R. Immunoregulation in the tissues by γδ T cells. Nat Rev Immunol (2003) 3(3):233-42. doi:10.1038/nri1030
8—Behr C, Poupot R, Peyrat M A, Poquet Y, Constant P, Dubois P, et al. Plasmodium falciparum stimuli for human γδ T cells are related to phosphorylated antigens of mycobacteria. Infect Immun (1996) 64(8):2892-6.
9—Sandstrom A, Peigné C M, Léger A, Crooks J E, Konczak F, Gesnel M C, et al. The intracellular B30.2 domain of butyrophilin 3A1 binds phosphoantigens to mediate activation of human Vγ9Vδ2 T cells. Immunity (2014) 40(4):490-500. doi:10.1016/j.immuni.2014.03.003
10—Poggi A, Venturino C, Catellani S, Clavio M, Miglino M, Gobbi M, et al. Vδ1 T lymphocytes from B-CLL patients recognize ULBP3 expressed on leukemic B cells and up-regulated by trans-retinoic acid. Cancer Res (2004) 64(24):9172-9. doi:10.1158/0008-5472.CAN-04-2417.
11—Vitale M, Bottino C, Sivori S, et al. NKp44, a novel triggering surface molecule specifically expressed by activated natural killer cells, is involved in non-major histocompatibility complex-restricted tumor cell lysis. J Exp Med. 1998; 187(12):2065-2072. doi:10.1084/jem.187.12.2065.
12—Dieli, F., Poccia, F., Lipp, M., Sireci, G., Caccamo, N., Sano, C. D., & Salerno, A. (2003). Differentiation of Effector/Memory Vδ2 T Cells and Migratory Routes in Lymph Nodes or Inflammatory Sites. The Journal of Experimental Medicine, 198(3), 391-397. https://doi.org/10.1084/jem.20030235
13—Braakman E, Sturm E, Vijverberg K, van Krimpen B A, Gratama J W, Bolhuis R L. Expression of CD45 isoforms by fresh and activated human gamma delta T lymphocytes and natural killer cells. Int Immunol. 1991 July; 3(7):691-7. doi: 10.1093/intimm/3.7.691. PMID: 1832950.

1. An in-vitro method for transferring one or more nucleic acids sequences comprising one or more transgenes into γδ T cells with a pseudotyped retroviral vector particle or a virus-like particle thereof, comprising the steps:
a) activating γδ T cells in a sample of leukocytes by adding to said sample an aminobisphosphonate together with one or more cytokine(s) selected from the group consisting of IL-1β, IL-2, IL-7, IL-15, IL-18 and IL-21,
b) contacting said pseudotyped retroviral vector particle or virus-like particle thereof with said activated γδ T cells, wherein said pseudotyped retroviral vector particle or virus-like particle thereof comprises a modified baboon endogenous retrovirus (BaEV) envelope glycoprotein that binds specifically to and fuses with the surface membrane of hematopoietic cells, thereby genetically modifying said γδ T cells of said sample, and
c) expanding said genetically modified γδ T cells,
wherein said pseudotyped retroviral vector particle or virus-like particle thereof comprises at least:
a chimeric envelope glycoprotein which comprises or consists of a fusion of the transmembrane and extracellular domain of a baboon endogenous retrovirus (BaEV) envelope glycoprotein and the cytoplasmic tail domain of a murine leukemia virus (MLV) envelope glycoprotein; or a modified BaEV envelope glycoprotein wherein the cytoplasmic tail domain is devoid of a fusion inhibitory R peptide,
wherein at least 75% of the γδ T cells obtained following step (c) are CD45RA negative.

2. The method of claim 1, further comprising removing αβ T cells from the sample of leukocytes before step (a).

3. The method according to claim 1, wherein said aminobisphosphonate is zoledronic acid or pamidronate.

4. The method according to claim 1, wherein said activation of γδ T cells is performed by adding to said γδ T cells an aminobisphosphonate together with cytokines are IL-2 and IL-15.

5. The method according to claim 1, wherein said contacting said pseudotyped retroviral vector particle or virus-like particle thereof with said activated γδ T cells, is at day 3 after activation of the γδ T cells.

6. The method according to claim 1, wherein the genetically modified γδ T cells expand at least 400-fold at day 14 of the expansion.

7. A composition that comprises γδ T cells transduced with a pseudotyped retroviral vector particle comprising one or more transgenes,
wherein at least 75% of said γδ T cells are CD45RA negative,
wherein said pseudotyped retroviral vector particle comprises a modified baboon endogenous retrovirus (BaEV) envelope glycoprotein that binds specifically to and fuses with the surface membrane of hematopoietic cells, and
wherein said pseudotyped retroviral vector particle or virus-like particle thereof comprises at least: a chimeric envelope glycoprotein which comprises or consists of a fusion of the transmembrane and extracellular domain of a baboon endogenous retrovirus (BaEV) envelope glycoprotein and the cytoplasmic tail domain of a murine leukemia virus (MLV) envelope glycoprotein; or a modified BaEV envelope glycoprotein wherein the cytoplasmic tail domain is devoid of a fusion inhibitory R peptide.

8. (canceled)

9. The composition according to claim 7, wherein said one or more transgenes is a chimeric antigen receptor (CAR) or an exogenous T cell receptor (TCR).

10. (canceled)

11. The composition according to claim 7, wherein said one or more transgenes is a CAR that comprises:
a) an antigen binding domain specific for an antigen expressed on the surface of a target cell;
b) a transmembrane domain; and
c) an intracellular signaling domain.

12. The composition according to claim 7, wherein said one or more transgenes is a CAR that comprises:
a) an antigen binding domain specific for a soluble antigen;
b) a transmembrane domain; and
c) an intracellular signaling domain.

13. The composition according to claim 7, wherein said one or more transgenes is a CAR that comprises:
a) an antigen binding domain specific for a tag of a tagged polypeptide;
b) a transmembrane domain; and
c) an intracellular signaling domain.

14. The composition according to claim 13, which further comprises said tagged polypeptide, which binds specifically to an antigen expressed on the surface of a target cell or binds specifically to a soluble antigen.

15. The method of claim 1, wherein said sample of leukocytes is a whole blood sample, a leukapheresis, a buffy coat or a sample of peripheral blood mononuclear cells (PBMC).

16. The method of claim 1, wherein said pseudotyped retroviral vector particle or virus-like particle thereof is used in step (b) at a concentration of 0.1 to 1 multiplicity of infection (MOI).

17. The method of claim 1, wherein the expanding in step (c) is done in the absence of feeder cells, human serum, and an aminobisphosphonate, and in the presence of IL-2 and IL-15.


SEQUENCE LISTING (3 sequences)

SEQ ID NO: 1 - protein, 563 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 2 - protein, 546 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 3 - protein, 562 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVVQALVLTQQYHQLKPLEYEP